SEQUENCE LISTING (56 sequences)

SEQ ID NO: 1 - protein, 876 aa, Geobacillus sp.
MKKKLVLIDGNSVAYRAFFALPLLHNDKGVHTNAVYGFAMMLRKIVAEEAPTHLLVAFDAGKTTFRHEVFREYKGGRQQTPPELSEQFPLLRELLNAYRIPAYELDNYEADDIIGTLAARAEQAGFEVKVISGDRDLTQLASPHVTVEITKKGITEMESYTPETIREKYGLAPEQIVDLKGLMGDKSDNIPGVPGIGEKTAVKLLKQFGTVENVLDSIDEIQGEKLKETLRQHRDTALLSKRLAGICRDAPIALSLEDTAYEGEDRESVIALFKELGFQSLLEKMEGPRAEESEPLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQAFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLSARLKEERLQARLLLQVHDELILEAPSDEIERLCRIVPEVMEQAVTLRVPLKVDYHYGPTWYDAK

SEQ ID NO: 2 - protein, 295 aa, Synthetic construct
MKKKLVLIDGNSVAYRAFFALPLLHNDKGVHTNAVYGFAMMLRKIVAEEAPTHLLVAFDAGKTTFRHEVFREYKGGRQQTPPELSEQFPLLRELLNAYRIPAYELDNYEADDIIGTLAARAEQAGFEVKVISGDRDLTQLASPHVTVEITKKGITEMESYTPETIREKYGLAPEQIVDLKGLMGDKSDNIPGVPGIGEKTAVKLLKQFGTVENVLDSIDEIQGEKLKETLRQHRDTALLSKRLAGICRDAPIALSLEDTAYEGEDRESVIALFKELGFQSLLEKMEGPRAEESEP

SEQ ID NO: 3 - protein, 11 aa, Synthetic construct
MHHHHHHHHHH

SEQ ID NO: 4 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQAFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLSARLKEERLQARLLLQVHDELILEAPSDEIERLCRIVPEVMEQAVTLRVPLKVDYHYGPTWYDAK

SEQ ID NO: 5 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRRDTKKVHTIFNQALTQTGRLSSTEPNLQNIPIRLEEGRKIRQAFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLSARLKEERLQARLLLQVHDELILEAPSDEIERLCRIVPEVMEQAVTLRVPLKVDYHYGPTWYDAK

SEQ ID NO: 6 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQTFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPDITSRNFNVRSFAERTAMNTPIQGSAADIIKKAMIDLSARLKEERLQARLLLQVHDELILEAPSDEIERLCRIVPEVMEQAVTLRVPLKVDYHYGPTWYDAK

SEQ ID NO: 7 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQAFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPQITSRNFNVRSFAERTAMNTPIQGSAADIIKKAMIDLSARLKEERLQARLLLQVHDELILEAPSDEIERLCRIVPEVMEQAVTLRVPLKVDYHYGPTWYDAK

SEQ ID NO: 8 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQAFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPDITSRNFNVRSFAERTAINTPIQGSAADIIKKAMIDLSARLKEERLQARLLLQVHDELILEAPSDEIERLCRIVPEVMEQAVTLRVPLKVDYHYGPTWYDAK

SEQ ID NO: 9 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQTFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPDITSRNFNVRSFAERTAINTPIQGSAADIIKKAMIDLSARLKEERLQARLLLQVHDELILEAPSDEIERLCRIVPEVMEQAVTLRVPLKVDYHYGPTWYDAK

SEQ ID NO: 10 - protein, 592 aa, Synthetic construct
MHHHHHHHHHHLAHIDFTMAECVTEEMLADKAALVVEVVEDNYHDAPIVGIAVVSERGRFFLPSEVALADPQFRAWLADETKKKSMFDSKRATVALRWKGIDLRGVSFDLLLAAYLLNPGQDASDVAAVAKMKQYEAVRSDEAVYGKGAKRSVPDEPALAEHLVRKAAAIWALERPMMDDLRRNEQDELLMALEQPLAAILADMEFTGVKVDTERLNQMGQELAEQLRAAERRIYELAGQEFNINSPKQLGVILFEKLQLPVLKKTKTGYSTSADVLEKLAPHHEIVENILHYRQLGKLQSTYVEGLLKVVRPDTKKVHTIFNQALTQTGRLSSTEPNLQNIPIRLEEGRKIRQTFVPSEPGWLMFAADYSQIELRVLAHIAEDDNLIEAFRRDLDIHTKTAMDIFHVKEEDVTPNMRRQAKAVNFGIVYGISDYGLAQNLNITRKEAAEFIERYFASFPGVKRYMETIVQEAKQKGYVTTLLHRRRYLPQITSRNFNVRSFAERTAINTPIQGSAADIIKKAMIDLSARLKEERLQARLLLQVHDELILEAPSDEIERLCRIVPEVMEQAVTLRVPLKVDYHYGPTWYDAK

SEQ ID NO: 11 - DNA, 20 bp, Synthetic construct
  source 1..20: mol_type = other DNA
tgtcatgggatccggatgtt

SEQ ID NO: 12 - DNA, 20 bp, Synthetic construct
  source 1..20: mol_type = other DNA
caatagagccgctctcagag

SEQ ID NO: 13 - DNA, 19 bp, Synthetic construct
  source 1..19: mol_type = other DNA
ccagagaggaggttgccaa

SEQ ID NO: 14 - DNA, 19 bp, Synthetic construct
  source 1..19: mol_type = other DNA
tgcaggatgcagcgcctta

SEQ ID NO: 15 - DNA, 41 bp, Synthetic construct
  source 1..41: mol_type = other DNA
gcccaaacaacgacgatcggtaaaaccagcatccgtagcct

SEQ ID NO: 16 - DNA, 40 bp, Synthetic construct
  source 1..40: mol_type = other DNA
gcacgttctccaacggtgctggttgcttgttcagcgaact

SEQ ID NO: 17 - DNA, 19 bp, Synthetic construct
  source 1..19: mol_type = other DNA
gcgcgatcaaaacaacgtc

SEQ ID NO: 18 - DNA, 18 bp, Synthetic construct
  source 1..18: mol_type = other DNA
ggcccagttcctaggtag

SEQ ID NO: 19 - DNA, 19 bp, Synthetic construct
  source 1..19: mol_type = other DNA
gtgagagcggtgaaccaag

SEQ ID NO: 20 - DNA, 23 bp, Synthetic construct
  source 1..23: mol_type = other DNA
tccaattaacaccaatagcagtc

SEQ ID NO: 21 - DNA, 48 bp, Synthetic construct
  source 1..48: mol_type = other DNA
aaggtcttccttgccatgttgacccaaggtttacccaataatactgcg

SEQ ID NO: 22 - DNA, 41 bp, Synthetic construct
  source 1..41: mol_type = other DNA
aaattccctcgaggacaaggcgtagctcttcggtagtagcc

SEQ ID NO: 23 - DNA, 25 bp, Synthetic construct
  source 1..25: mol_type = other DNA
tgggttttacaaacctgtgatggct

SEQ ID NO: 24 - DNA, 20 bp, Synthetic construct
  source 1..20: mol_type = other DNA
gcacacggcgttcacccaac

SEQ ID NO: 25 - skipped

SEQ ID NO: 26 - DNA, 17 bp, Synthetic construct
  source 1..17: mol_type = other DNA
ccacggcgtgcaaagga

SEQ ID NO: 27 - DNA, 19 bp, Synthetic construct
  source 1..19: mol_type = other DNA
ctgacgagtaccggcgtct

SEQ ID NO: 28 - DNA, 48 bp, Synthetic construct
  source 1..48: mol_type = other DNA
gccacggagatcaacttctcctgtacgaagaccctcgaggctatcctc

SEQ ID NO: 29 - DNA, 44 bp, Synthetic construct
  source 1..44: mol_type = other DNA
aggactcgccgtccactctgcttggaatctcaaagaggccctgg

SEQ ID NO: 30 - DNA, 21 bp, Synthetic construct
  source 1..21: mol_type = other DNA
tggttctcgcatcgatgaaga

SEQ ID NO: 31 - DNA, 23 bp, Synthetic construct
  source 1..23: mol_type = other DNA
cgtagatttttttcgtgcaagct

SEQ ID NO: 32 - DNA, 20 bp, Synthetic construct
  source 1..20: mol_type = other DNA
tcacgtctgcaagtcatact

SEQ ID NO: 33 - DNA, 18 bp, Synthetic construct
  source 1..18: mol_type = other DNA
tgcctgtttgagcgtgat

SEQ ID NO: 34 - DNA, 46 bp, Synthetic construct
  source 1..46: mol_type = other DNA
tgtgcgttcaaagattcgatgatttttgcagcgaaatgcgatacgt

SEQ ID NO: 35 - DNA, 51 bp, Synthetic construct
  source 1..51: mol_type = other DNA
ttgcgccttggggtattccccaagtttttttgtgaatgcaacgccaccgcg

SEQ ID NO: 36 - DNA, 58 bp, Synthetic construct
  source 1..58: mol_type = other DNA
gagggcctgctgaaagtcgtacgtcgcgacaccaaaaaagtccatactatctttaacc

SEQ ID NO: 37 - DNA, 58 bp, Synthetic construct
  source 1..58: mol_type = other DNA
ggttaaagatagtatggacttttttggtgtcgcgacgtacgactttcagcaggccctc

SEQ ID NO: 38 - DNA, 44 bp, Synthetic construct
  source 1..44: mol_type = other DNA
gagggccgtaaaatccgccaaacgtttgttccgagcgagccggg

SEQ ID NO: 39 - DNA, 44 bp, Synthetic construct
  source 1..44: mol_type = other DNA
cccggctcgctcggaacaaacgtttggcggattttacggccctc

SEQ ID NO: 40 - DNA, 53 bp, Synthetic construct
  source 1..53: mol_type = other DNA
caccgtcgtcgttatctgccgcagatcactagtcgcaatttcaacgtccgtag

SEQ ID NO: 41 - DNA, 53 bp, Synthetic construct
  source 1..53: mol_type = other DNA
ctacggacgttgaaattgcgactagtgatctgcggcagataacgacgacggtg

SEQ ID NO: 42 - DNA, 45 bp, Synthetic construct
  source 1..45: mol_type = other DNA
cttcgcggagcgtacggcaattaatacgccgatccagggcagcgc

SEQ ID NO: 43 - DNA, 45 bp, Synthetic construct
  source 1..45: mol_type = other DNA
gcgctgccctggatcggcgtattaattgccgtacgctccgcgaag

SEQ ID NO: 44 - DNA, 30 bp, Synthetic construct
  source 1..30: mol_type = other DNA
tgtcatgggatccggatgttttacaaacca

SEQ ID NO: 45 - DNA, 21 bp, Synthetic construct
  source 1..21: mol_type = other DNA
acggcgcacattggtctcgga

SEQ ID NO: 46 - DNA, 24 bp, Synthetic construct
  source 1..24: mol_type = other DNA
cgccagggttttcccagtcacgac

SEQ ID NO: 47 - DNA, 2631 bp, Geobacillus sp.
  source 1..2631: mol_type = genomic DNA
ttgaagaaaaaacttgtcttgattgacggcaacagtgtggcctatcgcgctttttttgcgctgccgcttttgcataacgacaaaggcgttcatacgaatgcggtctacggatttgccatgatgctgcgtaaaatcgtggcggaagaggctcccactcatttgttagtggcgtttgatgcgggcaaaacgacatttcgtcatgaagtgtttcgcgagtataaaggcgggcgccagcagacgcccccggaattgtccgagcagtttccactgctgcgcgagctgttgaacgcctatcgcattccagcttacgaacttgacaactacgaagcggacgacattatcggaacgctcgccgcccgcgctgagcaggccgggtttgaggtgaaagtcatctccggagaccgggatttgacccaactcgcctccccccatgtgacggtggaaatcacgaaaaaagggatcaccgagatggaatcgtacacgccggaaacgattcgcgaaaaatacgggctcgctcccgagcaaatcgttgacttaaaaggactgatgggggataaatcggacaacatccctggcgtgcccggcatcggcgaaaaaacggcggtcaagttgttaaagcagttcgggacagtcgaaaatgtgctcgactccattgatgaaatccaaggggaaaagctgaaagaaacgctgcgccaacatcgcgataccgccctcctaagcaaacggctggcgggcatttgccgcgacgctccgatcgccctgtcgcttgaggatacggcctacgaaggcgaagaccgggagagcgtgatcgctttgtttaaagagctcgggtttcagtcgctccttgagaagatggaagggccgagggcggaagagagcgagccgcttgcccacatcgacttcaccatggcggagtgcgtgacggaagagatgctcgccgacaaggcggccttggtcgttgaagtcgtggaagacaactatcatgacgcgccgatcgtcggcattgctgttgtctcggaacgcgggcggtttttcctgccttcggaggtggcgctggctgacccgcagtttcgcgcttggcttgccgatgagacgaagaaaaaaagcatgtttgactcgaagcgggcgacggtcgctttacgatggaaagggatcgacctgcgcggcgtctcgtttgatttgctgctggcggcgtatttgctcaaccctggacaagatgccagcgatgtcgcggcggtggccaaaatgaaacaatacgaggccgtgcggtccgatgaagcggtctacggcaaaggggcgaaacggtctgttccggacgagccggcactcgccgagcatctcgtccgcaaggcggcggcgatttgggcgctcgagcggccaatgatggatgacttgcgccgcaacgagcaggacgaactgttgatggcgcttgagcagccgttagcggcgattttggccgatatggaattcaccggggtgaaagtggacaccgagcgcctgaaccagatgggacaggaactcgccgagcagctgcgcgcggccgaacggcgcatttacgaactggccggccaagaattcaatatcaattcgccgaaacagctcggcgtcattttatttgaaaagctgcaactgcctgtgttgaaaaaaacgaagaccggctattcaacatcggcggacgtgctggaaaagcttgcgccccatcacgagattgtggagaatattttgcactaccgccaactaggcaagctgcagtcgacatatgtcgaaggattgttgaaggtcgtgcgccccgatacgaagaaagtgcatacgatcttcaatcaagcgttgacgcaaaccggccgcctcagctcgacggagccgaacttgcaaaacattccgattcggctggaagaagggcggaaaatccggcaagcgttcgtgccgtccgagcctggctggctcatgtttgcggctgactattcgcaaatcgaactgcgcgtgttggcccatatcgccgaagatgacaatttaattgaagcgtttcgccgcgatttggacattcatacgaaaacggctatggacatttttcacgtcaaggaagaggacgtcacccccaacatgcgccggcaagcgaaggccgtcaactttggcatcgtctatgggatcagcgattacggattggcgcaaaacttaaacattacgcgcaaagaggccgccgagtttatcgaacgctattttgccagcttcccgggcgtcaagcggtatatggaaacgatcgtgcaagaggcgaaacagaaaggatatgtcaccacgcttttgcatcggcgccgctatttgccggacatcacgagccgcaacttcaacgtccgcagctttgccgagcggacggcgatgaatacgccgatccaaggcagcgccgctgacatcattaaaaaggcgatgatcgatttgagcgcgcgcctgaaggaagagcggctgcaggcgcgcctgttgctgcaagtgcacgacgagctcattttggaggcgcctagtgacgaaatagagcggctgtgccgaatcgtgccggaagtgatggagcaagccgtcacgctccgcgtgccgctgaaagtcgattatcattacggaccgacatggtatgatgcgaaataa

SEQ ID NO: 48 - DNA, 885 bp, Synthetic construct
  source 1..885: mol_type = other DNA
ttgaagaaaaaacttgtcttgattgacggcaacagtgtggcctatcgcgctttttttgcgctgccgcttttgcataacgacaaaggcgttcatacgaatgcggtctacggatttgccatgatgctgcgtaaaatcgtggcggaagaggctcccactcatttgttagtggcgtttgatgcgggcaaaacgacatttcgtcatgaagtgtttcgcgagtataaaggcgggcgccagcagacgcccccggaattgtccgagcagtttccactgctgcgcgagctgttgaacgcctatcgcattccagcttacgaacttgacaactacgaagcggacgacattatcggaacgctcgccgcccgcgctgagcaggccgggtttgaggtgaaagtcatctccggagaccgggatttgacccaactcgcctccccccatgtgacggtggaaatcacgaaaaaagggatcaccgagatggaatcgtacacgccggaaacgattcgcgaaaaatacgggctcgctcccgagcaaatcgttgacttaaaaggactgatgggggataaatcggacaacatccctggcgtgcccggcatcggcgaaaaaacggcggtcaagttgttaaagcagttcgggacagtcgaaaatgtgctcgactccattgatgaaatccaaggggaaaagctgaaagaaacgctgcgccaacatcgcgataccgccctcctaagcaaacggctggcgggcatttgccgcgacgctccgatcgccctgtcgcttgaggatacggcctacgaaggcgaagaccgggagagcgtgatcgctttgtttaaagagctcgggtttcagtcgctccttgagaagatggaagggccgagggcggaagagagcgagccg

SEQ ID NO: 49 - DNA, 33 bp, Synthetic construct
  source 1..33: mol_type = other DNA
atgcaccaccatcatcatcaccatcaccaccac

SEQ ID NO: 50 - DNA, 1779 bp, Synthetic construct
  source 1..1779: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgctgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtgcgtccggacaccaaaaaagtccatactatctttaaccaagcgctgacccagacgggtcgtcttagcagcaccgagccgaatctgcaaaacattccgatccgcttggaagagggccgtaaaatccgccaggcctttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccggacatcacgagccgcaatttcaacgtccgtagcttcgcggagcgtacggcaatgaacacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

SEQ ID NO: 51 - DNA, 1778 bp, Synthetic construct
  source 1..1778: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgcgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtacgtcgcgacaccaaaaaagtccatactatctttaaccaagcgctgacccagacgggtcgtcttagcagcaccgagccgaatctgcaaaacattccgatccgcttggaagagggccgtaaaatccgccaggcctttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccggacatcacgagccgcaatttcaacgtccgtagcttcgcggagcgtacggcaatgaacacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

SEQ ID NO: 52 - DNA, 1779 bp, Synthetic construct
  source 1..1779: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgctgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtgcgtccggacaccaaaaaagtccatactatctttaaccaagcgctgacccagacgggtcgtcttagcagcaccgagccgaatctgcaaaacattccgatccgcttggaagagggccgtaaaatccgccaaacgtttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccggacatcacgagccgcaatttcaacgtccgtagcttcgcggagcgtacggcaatgaacacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

SEQ ID NO: 53 - DNA, 1779 bp, Synthetic construct
  source 1..1779: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgctgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtgcgtccggacaccaaaaaagtccatactatctttaaccaagcgctgacccagacgggtcgtcttagcagcaccgagccgaatctgcaaaacattccgatccgcttggaagagggccgtaaaatccgccaggcctttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccgcagatcactagtcgcaatttcaacgtccgtagcttcgcggagcgtacggcaatgaacacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

SEQ ID NO: 54 - DNA, 1779 bp, Synthetic construct
  source 1..1779: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgctgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtgcgtccggacaccaaaaaagtccatactatctttaaccaagcgctgacccagacgggtcgtcttagcagcaccgagccgaatctgcaaaacattccgatccgcttggaagagggccgtaaaatccgccaggcctttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccggacatcacgagccgcaatttcaacgtccgtagcttcgcggagcgtacggcaattaatacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

SEQ ID NO: 55 - DNA, 1779 bp, Synthetic construct
  source 1..1779: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgctgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtgcgtccggacaccaaaaaagtccatactatctttaaccaagcgctgacccagacgggtcgtcttagcagcaccgagccgaatctgcaaaacattccgatccgcttggaagagggccgtaaaatccgccaaacgtttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccggacatcacgagccgcaatttcaacgtccgtagcttcgcggagcgtacggcaattaatacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

SEQ ID NO: 56 - DNA, 1779 bp, Synthetic construct
  source 1..1779: mol_type = other DNA
atgcaccaccatcatcatcaccatcaccaccacctggcccacatcgacttcaccatggccgagtgcgttaccgaagaaatgctggcggataaggcagcgctggtcgttgaagttgttgaggataactaccacgacgctccgattgtgggtattgcggtcgtgagcgagcgcggtcgcttcttcctgccgagcgaagtcgcgctggcggatccacagtttcgcgcatggctggcggatgagactaaaaagaaatccatgtttgatagcaagcgcgcgacggttgcgctgcgttggaagggtatcgatctgcgtggtgtgagcttcgatctgctcttggcggcttacttgctgaatccgggccaagacgcgtccgacgttgcagcggtggcgaagatgaaacagtacgaagctgtccgttcggatgaagccgtatatggtaagggtgcaaagcgcagcgtcccggatgagccggcattggcagagcatctggtgcgtaaagctgctgcgatttgggcgttggagcgtcctatgatggatgacttgcgccgtaacgaacaagacgaactgctgatggcgctggagcagccgctggcggcaatcctggccgacatggaattcaccggtgtcaaagttgacaccgagcgtctgaaccagatgggtcaagaattagcggagcaactgcgtgcagcggagcgcagaatctacgaattggcaggccaagaattcaacatcaacagcccgaaacagctgggtgtgattctgtttgaaaagttacagctgccggtcctgaagaaaactaagaccggctacagcacctctgcagacgtgttggagaaactggccccacaccacgaaatcgtggagaatatcctgcattaccgtcagctgggcaagctgcagagcacctatgttgagggcctgctgaaagtcgtgcgtccggacaccaaaaaagtccatactatctttaaccaagcgctgacccagacgggtcgtcttagcagcaccgagccgaatctgcaaaacattccgatccgcttggaagagggccgtaaaatccgccaaacgtttgttccgagcgagccgggttggctgatgtttgcggctgattatagccagatcgagttgcgtgtgctggcgcatattgcagaggacgacaatctgattgaagcctttcgtcgcgatctggatattcatacgaaaaccgccatggacatcttccacgtcaaagaggaagatgtgaccccgaacatgcgtcgtcaggcgaaagctgttaatttcggtattgtttacggcatttctgactatggtctggcccagaatctgaatattacgcgtaaagaggccgcagagtttattgagcgttatttcgcgtctttcccgggtgtcaagcgttacatggaaaccattgtgcaagaagcgaagcaaaagggctatgtgacgaccctgttgcaccgtcgtcgttatctgccgcagatcactagtcgcaatttcaacgtccgtagcttcgcggagcgtacggcaattaatacgccgatccagggcagcgcagcggacatcatcaaaaaagccatgatcgacctgagcgcacgcctgaaagaggaacgcctgcaggcccgtctgctgctgcaagtgcacgatgaactgattctggaagcgccgtccgatgaaattgagcgtctgtgtcgcattgttcctgaagttatggaacaagctgtgaccctccgtgttccgctgaaagttgactaccactacggtccgacctggtatgacgcaaagtga

(19) United States
(12) Patent Application Publication
(10) Pub. No.: US 2025/0382591 A1
(43) Pub. Date: Dec. 18, 2025

(54) BST POLYMERASES WITH REVERSE TRANSCRIPTASE ACTIVITY

(71) Applicant: Varigen Biosciences dba Varizymes, MIddleton, WI (US)

(72) Inventors: Yogesh Chander, Waunakee, WI (US); Kurt Throckmorton, Middleton, WI (US); Aaron Lomax, Madison, WI (US); Alyssa Hassinger, Madison, WI (US); David A. Mead, Middleton, WI (US)

(21) Appl. No.: 19/240,518

(22) Filed: Jun. 17, 2025

Related U.S. Application Data
(60) Provisional application No. 63/661,364, filed on Jun. 18, 2024.

(51) Int. Cl. C12N 9/12; C12Q 1/6844

(52) U.S. Cl. CPC C12N 9/1276; C12Q 1/6844; C12Y 207/07049

(57) ABSTRACT

Provided herein are compositions and methods for use in amplification assays. In particular, provided herein are Bst polymerases with enhanced reverse transcriptase (RT) activity for use in amplification assays such as, for example, loop-mediated isothermal amplification (LAMP) assays.

### CROSS-REFERENCE TO RELATED APPLICATIONS

This application claims priority to and the benefit of U.S. Provisional Patent Application No. 63/661,364, filed Jun. 18, 2024, which is incorporated herein by reference in its entirety.

### SEQUENCE LISTING

The text of the computer readable sequence listing filed herewith, titled “VARI_43282_202_SequenceListing.xml”, created Jun. 16, 2025, having a file size of 70,999 bytes, is hereby incorporated by reference in its entirety.

### FIELD

Provided herein are compositions and methods for use in amplification assays. In particular, provided herein are Bst polymerases with enhanced reverse transcriptase (RT) activity for use in amplification assays such as, for example, loop-mediated isothermal amplification (LAMP) assays.

### BACKGROUND

LAMP (loop-mediated isothermal amplification) is an attractive alternative to polymerase chain reaction (PCR) for detection of nucleic acid (DNA and/or RNA) (Mori et al, Loop-mediated isothermal amplification (LAMP): a rapid, accurate, and cost-effective diagnostic method for infectious diseases. J Infect Chemother. 2009; 15:62-69). Use of LAMP is advantageous as it is simple, rapid, and cost-effective as compared to conventional PCR or other PCR-based assays (Francois et al., Robustness of a loop-mediated isothermal amplification reaction for diagnostic applications. FEMS Immunol Med Microbiol. 2011; 62:41-48.). The sensitivity and specificity of LAMP are comparable to PCR, and because LAMP amplification is carried out at a constant temperature and does not require expensive thermocycler instrumentation, this makes it easy to use for resource-limited laboratories. Bst DNA polymerases (DNAPs) are commonly used for amplification of DNA sequences in a LAMP assay. For detection of RNA sequences, LAMP reaction formulations are usually supplemented with an additional reverse transcriptase (RT) enzyme along with Bst DNAP. While many Bst DNAPs have minimal RT activity, this is not enough to achieve high sensitivity and LAMP assays are usually supplemented with RT enzymes such as HIV RT, MMLV, mAMV, etc.

A Bst DNAP with enhanced RT activity would overcome the need to use additional RT enzymes in LAMP assays.

### SUMMARY

Provided herein are compositions and methods for use in amplification assays. In particular, provided herein are Bst polymerases with enhanced reverse transcriptase (RT) activity for use in amplification assays such as, for example, loop-mediated isothermal amplification (LAMP) assays.

The present disclosure provides variant Bst polymerases with increased RT activity. Such polymerases provide improved function in amplification assays and cost savings by eliminating the need for supplemental RT polymerases in such assays. For example, in some embodiments, provided herein is a variant Bst polypeptide having an amino acid sequence selected from, for example, SEQ ID Nos: 5-10.

Also provided is a variant Bst polypeptide having at least one mutation of the amino acid sequence shown in SEQ ID NO: 1, wherein the mutation is one or more (e.g., 1, 2, or 3) of P597R, A639T, D775Q, or M792I.

In some embodiments, the variant exhibits increased reverse transcriptase activity relative to wild type Bst (e.g., an increase of at least 10%, 20%, 50%, 100%, 200%, or more).

In some embodiments, the polypeptide lacks an exonuclease domain.

Further provided is a composition, kit, or reaction mixture comprising a variant polypeptide described herein. In some embodiments, the composition, kit, or reaction mixture comprises one or more buffers (e.g., including but not limited to, Tris-HCl, (NH₄)₂SO₄, KCl, MgSO₄, and Triton® X-100 detergent). In some embodiments, the composition, kit or reaction mixture further comprises a RT enzyme. In some embodiments, the composition, kit or reaction mixture further comprises one or more additional components selected from, for example, nucleic acid primers, probes, dNTPs, dyes, ions, or nucleic acid controls. In some embodiments, the pH of composition, kit or reaction mixture is between 8 and 9 (e.g., 8.8).

Also provided are nucleic acids encoding polypeptides described herein. In some embodiments, the nucleic acid has a sequence selected from SEQ ID Nos: 51-56.

Certain embodiments provide a method of amplifying a target nucleic acid, comprising: a) contacting a sample with a polypeptide, composition, kit, or reaction mixture described herein; and b) performing an amplification assay on the sample.

Additional embodiments provide a method of detecting a target nucleic acid, comprising: a) contacting a sample with a polypeptide, composition, kit, or reaction mixture described herein; and b) performing an amplification assay on the sample.

Further provided herein is the use of a polypeptide, composition, kit, or reaction mixture described herein to perform an amplification assay or polypeptide, composition, kit, or reaction mixture described herein for use in performing an amplification assay.

The present disclosure is not limited to a particular amplification assay. In some examples, the amplification assay is an isothermal amplification assay (e.g., loop-mediated isothermal amplification (LAMP)).

Additional embodiments are described herein.

### BRIEF DESCRIPTION OF THE DRAWINGS

FIGS. 1A-1B: Graphical depiction of reverse transcriptase (RT) and DNA-dependent DNA polymerase activities of Bst496 DNA polymerase (DNAP) and its variants in comparison with Bst 2.0 WarmStart (WS) and Bst 3.0. FIG. 1A depicts RT activity of WT-Δexo-Bst496 and its variants, relative to Bst 2.0 WS and 3.0 (New England Biolabs, MA). FIG. 1B depicts the DNA-dependent DNAP activity of WT-Δexo-Bst496 and its variants, relative to Bst 2.0 WS and 3.0 (New England Biolabs, MA).

FIGS. 2A-2B: Graphical depiction of performance of WT-Δexo-Bst496 DNA polymerase (DNAP), its variants, and Bst 2.0 WarmStart (WS) and Bst 3.0 (New England Biolabs, MA) in a RT-LAMP assay for amplification of MS2 RNA. The patterns of the bars represents the MS2 RNA concentration in ug per reaction. FIG. 2A shows data for MS2 LAMP with different Bst DNAPs without additional RT. FIG. 2B shows data for MS2 LAMP with different DNAPs with additional RT.

FIGS. 3A-3B: Graphical depiction of performance of WT-Δexo-Bst496 DNA polymerase (DNAP), its variants, and Bst 2.0 WarmStart (WS) and Bst 3.0 (New England Biolabs, MA) in a RT-LAMP assay for amplification of SARS-COV-2 RNA. The patterns of the bars represents the SARS-COV-2 RNA concentration in copies per reaction. FIG. 3A shows data for SARS-COV-2 LAMP with different Bst DNAPs without additional RT. FIG. 3B shows data for SARS-COV-2 LAMP with different Bst DNAPs with additional RT.

FIGS. 4A-4B: Graphical depiction of performance of WT-Δexo-Bst496 DNA polymerase (DNAP), its variants, and Bst 2.0 WarmStart (WS) and Bst 3.0 (New England Biolabs, MA) in a RT-LAMP assay for amplification of Foot and Mouth disease virus (FMDV) VariSafe™ RNA control (Varizymes, Middleton, WI). The patterns of the bars represents the FMDV VariSafe RNA concentration in copies per reaction. FIG. 4A shows data for FMDV LAMP with different Bst DNAPs without additional RT. FIG. 4B shows data for FMDV LAMP with different Bst DNAPs with additional RT.

FIG. 5: Graphical depiction of performance of WT-Δexo-Bst496 DNA polymerase (DNAP), its variants, and Bst 2.0 WS and Bst 3.0 (New England Biolabs, MA) in a LAMP assay for amplification of Candida auris DNA. The patterns of the bars represents the C. auris DNA concentration in picograms per reaction.

FIGS. 6A-6B: Graphical depiction of melt curve for WT-Δexo-Bst496 DNA polymerase (DNAP), its variants (FIG. 6B), and Bst 2.0 WarmStart (WS) and Bst 3.0 (New England Biolabs, MA) (FIG. 6A). The reactions were performed using Protein Thermal Shift Dye Kit™ (Thermo Fisher; catalog #4461146).

FIG. 7: Reverse transcriptase (RT) activity of WT-Δexo-Bst496 DNA polymerase (DNAP) and its variants at different temperatures.

### DETAILED DESCRIPTION

Provided herein are compositions and methods for use in amplification assays. In particular, provided herein are Bst polymerases with enhanced reverse transcriptase (RT) activity for use in amplification assays such as, for example, loop mediated isothermal amplification (LAMP) assays.

In some embodiments, the present disclosure relates to the description of a Bst DNAP, WT-Δexo-Bst496 and its variants suitable for robust amplification of a target RNA and DNA sequence in a LAMP reaction. In some embodiments, this disclosure describes WT-Δexo-Bst496 DNAP and its variants with improved RT activity in addition to DNA polymerase activity. In certain embodiments, Bst496 DNAP (e.g., WT-Δexo-Bst496 DNAP) variants can be used for amplification of RNA in a LAMP reaction without the use of additional enzymes (RT).

As used herein, a “variant” or “mutant” Bst DNAP refers to a Bst DNAP that differs from parent or wild-type (WT) Bst DNAP by at least one amino acid. The variants may share >95% homology to the WT Bst DNAP as determined by any method known in the art such as BLAST. In addition, variants may have one or more improved properties as compared with the WT such as faster time to results, improved RT activity, thermostability, and tolerance to inhibitors. In some embodiments, the variant Bst DNAP is a Bst DNAP described by one of SEQ ID SOs: 5-10.

### Bst Polymerase Variants

The present disclosure is not limited to particular Bst polymerase variants. Any number of suitable variants may be utilized. While certain aspects of the disclosure are illustrated with Bst496 DNAP and variants thereof, additional DNAPs are specifically contemplated herein. In certain embodiments, variants have increased RT activity relative to WT Bst polymerases. In some embodiments, the Bst polymerase variants of the present disclosure comprise one or more mutations relative to a full-length, wild-type Geobacillus sp. C56-T2 (Genbank Accession No. TWG31496.1) Bst polymerase (SEQ ID NO: 1), alternatively referred to as “FL-WT-Bst496” herein. Thus, the Bst polymerase variants of the disclosure may be alternately referred to herein as “Bst496 polymerase variants.”

In some embodiments, a Bst polymerase variant comprises one or more modifications to the full-length, wild-type Bst nucleic acid polymerase having an amino acid sequence as shown in SEQ ID NO: 1. In some embodiments, such modifications optimize the full-length, wild-type Bst polymerase for certain amplification (e.g., LAMP, RT-LAMP) and/or purification (e.g., Ni-affinity column protein purification) methods. In some embodiments, the 5′ to 3′ exonuclease domain of full-length, wild-type Bst polymerase (Geobacillus sp. C56-T2 (Genbank Accession No. TWG31496.1); SEQ ID NO: 1), located at the N-terminus of the wild-type protein, is deleted. In some embodiments, the 5′ to 3′ exonuclease domain of full-length, wild-type Bst496 polymerase (Geobacillus sp. C56-T2 (Genbank Accession No. TWG31496.1); SEQ ID NO: 1) comprises an amino acid sequence as shown in SEQ ID NO: 2. In some embodiments, a ten-histidine tag is added to the N-terminus of the 5′ to 3′ exonuclease domain-deficient Bst496 polymerase. In some embodiments, the ten-histidine tag comprises an amino acid sequence as shown in SEQ ID NO: 3. Accordingly, in some embodiments the Bst polymerase variant comprises an amino acid sequence which does not comprise a 5′ to 3′ exonuclease domain and which does comprise a ten-histidine tag at its N-terminus, relative to full-length, wild-type Bst496 polymerase (Geobacillus sp. C56-T2 (Genbank Accession No. TWG31496.1); SEQ ID NO: 1). In some embodiments, said Bst polymerase variant comprises an amino acid sequence as shown in SEQ ID NO: 4. The Bst polymerase variant which comprises an amino acid sequence as shown in SEQ ID NO: 4 is referred to herein as an “WT-Δexo-Bst496” polymerase.

In some embodiments, a Bst polymerase variant of the disclosure comprises one or more amino acid mutations, relative to the full-length, wild-type Bst496 polymerase sequence (SEQ ID NO: 1) and/or to the WT-Δexo-Bst496 polymerase sequence (SEQ ID NO: 4). In some embodiments, a Bst polymerase variant of the disclosure comprises a plurality (e.g., 1, 2, or 3, etc.) amino acid mutations, relative to the full-length, wild-type Bst496 polymerase sequence (SEQ ID NO: 1) and/or the WT-Δexo-Bst496 polymerase sequence (SEQ ID NO: 4). As will be understood, an amino acid mutation may comprise the addition, deletion, or substitution (e.g., a substitution with a hydrophobic amino acid, for example A, L, or V, a substitution with a polar amino acid, for example N, S, or Q, or other amino acid substitution) of an amino acid. Any amino acid mutation described herein may be made alone or in combination, without limitation.

TABLE 1
Amino acid sequences of FL-WT-Bst496 DNA polymerase (DNAP) and its
variants

| SEQ | | |
|---|---|---|
| ID NO | Description | Sequence |
| 1 | FL-WT- | MKKKLVLIDGNSVAYRAFFALPLLHNDKGVHTNAVYGFAMML |
| | Bst496, | RKIVAEEAPTHLLVAFDAGKTTFRHEVFREYKGGRQQTPPELSE |
| | TWG31496 | QFPLLRELLNAYRIPAYELDNYEADDIIGTLAARAEQAGFEVKVI |
| | | SGDRDLTQLASPHVTVEITKKGITEMESYTPETIREKYGLAPEQIV |
| | | DLKGLMGDKSDNIPGVPGIGEKTAVKLLKQFGTVENVLDSIDEIQ |
| | | GEKLKETLRQHRDTALLSKRLAGICRDAPIALSLEDTAYEGEDRE |
| | | SVIALFKELGFQSLLEKMEGPRAEESEPLAHIDFTMAECVTEEML |
| | | ADKAALVVEVVEDNYHDAPIVGIAVVSERGRFFLPSEVALADPQ |
| | | FRAWLADETKKKSMFDSKRATVALRWKGIDLRGVSFDLLLAAY |
| | | LLNPGQDASDVAAVAKMKQYEAVRSDEAVYGKGAKRSVPDEP |
| | | ALAEHLVRKAAAIWALERPMMDDLRRNEQDELLMALEQPLAAI |
| | | LADMEFTGVKVDTERLNQMGQELAEQLRAAERRIYELAGQEFN |
| | | INSPKQLGVILFEKLQLPVLKKTKTGYSTSADVLEKLAPHHEIVE |
| | | NILHYRQLGKLQSTYVEGLLKVVRPDTKKVHTIFNQALTQTGRL |
| | | SSTEPNLQNIPIRLEEGRKIRQAFVPSEPGWLMFAADYSQIELRVL |
| | | AHIAEDDNLIEAFRRDLDIHTKTAMDIFHVKEEDVTPNMRRQAK |
| | | AVNFGIVYGISDYGLAQNLNITRKEAAEFIERYFASFPGVKRYME |
| | | TIVQEAKQKGYVTTLLHRRRYLPDITSRNFNVRSFAERTAMNTPI |
| | | QGSAADIIKKAMIDLSARLKEERLQARLLLQVHDELILEAPSDEIE |
| | | RLCRIVPEVMEQAVTLRVPLKVDYHYGPTWYDAK |
| 2 | Exo, | MKKKLVLIDGNSVAYRAFFALPLLHNDKGVHTNAVYGFAMML |
| | exonuclease | RKIVAEEAPTHLLVAFDAGKTTFRHEVFREYKGGRQQTPPELSE |
| | domain | QFPLLRELLNAYRIPAYELDNYEADDIIGTLAARAEQAGFEVKVI |
| | | SGDRDLTQLASPHVTVEITKKGITEMESYTPETIREKYGLAPEQIV |
| | | DLKGLMGDKSDNIPGVPGIGEKTAVKLLKQFGTVENVLDSIDEIQ |
| | | GEKLKETLRQHRDTALLSKRLAGICRDAPIALSLEDTAYEGEDRE |
| | | SVIALFKELGFQSLLEKMEGPRAEESEP |
| 3 | 10XHis, tag | MHHHHHHHHHH |
| | for nickel | |
| | affinity | |
| | purification | |
| 4 | WT-Δexo- | MHHHHHHHHHHLAHIDFTMAECVTEEMLADKAALVVEVVEDN |
| | Bst496, | YHDAPIVGIAVVSERGRFFLPSEVALADPQFRAWLADETKKKSM |
| | 10XHis Aexo | FDSKRATVALRWKGIDLRGVSFDLLLAAYLLNPGQDASDVAAV |
| | Bst496 | AKMKQYEAVRSDEAVYGKGAKRSVPDEPALAEHLVRKAAAIW |
| | | ALERPMMDDLRRNEQDELLMALEQPLAAILADMEFTGVKVDTE |
| | | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRPDTKKVHTIFNQALTQTGRLSSTEPNLQNIPIRLEE |
| | | GRKIRQAFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPDITSRNFNVRSFAERTAMNTPIQGSAADIIKKAMID |
| | | LSARLKEERLQARLLLQVHDELILEAPSDEIERLCRIVPEVMEQA |
| | | VTLRVPLKVDYHYGPTWYDAK |
| 5 | Bst496 | MHHHHHHHHHHLAHIDFTMAECVTEEMLADKAALVVEVVEDN |
| | P597R | YHDAPIVGIAVVSERGRFFLPSEVALADPQFRAWLADETKKKSM |
| | | FDSKRATVALRWKGIDLRGVSFDLLLAAYLLNPGQDASDVAAV |
| | | AKMKQYEAVRSDEAVYGKGAKRSVPDEPALAEHLVRKAAAIW |
| | | ALERPMMDDLRRNEQDELLMALEQPLAAILADMEFTGVKVDTE |
| | | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRRDTKKVHTIFNQALTQTGRLSSTEPNLQNIPIRLEE |
| | | GRKIRQAFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPDITSRNFNVRSFAERTAMNTPIQGSAADIIKKAMID |
| | | LSARLKEERLQARLLLQVHDELILEAPSDEIERLCRIVPEVMEQA |
| | | VTLRVPLKVDYHYGPTWYDAK |
| 6 | Bst496 | MHHHHHHHHHHLAHIDFTMAECVTEEMLADKAALVVEVVEDN |
| | A639T | YHDAPIVGIAVVSERGRFFLPSEVALADPQFRAWLADETKKKSM |
| | | FDSKRATVALRWKGIDLRGVSFDLLLAAYLLNPGQDASDVAAV |
| | | AKMKQYEAVRSDEAVYGKGAKRSVPDEPALAEHLVRKAAAIW |
| | | ALERPMMDDLRRNEQDELLMALEQPLAAILADMEFTGVKVDTE |
| | | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRPDTKKVHTIFNQALTQTGRLSSTEPNLQNIPIRLEE |
| | | GRKIRQTFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPDITSRNFNVRSFAERTAMNTPIQGSAADIIKKAMID |
| | | LSARLKEERLQARLLLQVHDELILEAPSDEIERLCRIVPEVMEQA |
| | | VTLRVPLKVDYHYGPTWYDAK |
| 7 | Bst496 | MHHHHHHHHHHLAHIDFTMAECVTEEMLADKAALVVEVVEDN |
| | D775Q | YHDAPIVGIAVVSERGRFFLPSEVALADPQFRAWLADETKKKSM |
| | | FDSKRATVALRWKGIDLRGVSFDLLLAAYLLNPGQDASDVAAV |
| | | AKMKQYEAVRSDEAVYGKGAKRSVPDEPALAEHLVRKAAAIW |
| | | ALERPMMDDLRRNEQDELLMALEQPLAAILADMEFTGVKVDTE |
| | | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRPDTKKVHTIFNQALTQTGRLSSTEPNLQNIPIRLEE |
| | | GRKIRQAFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPQITSRNFNVRSFAERTAMNTPIQGSAADIIKKAMID |
| | | LSARLKEERLQARLLLQVHDELILEAPSDEIERLCRIVPEVMEQA |
| | | VTLRVPLKVDYHYGPTWYDAK |
| 8 | Bst496 | MHHHHHHHHHHLAHIDFTMAECVTEEMLADKAALVVEVVEDN |
| | M792I | YHDAPIVGIAVVSERGRFFLPSEVALADPQFRAWLADETKKKSM |
| | | FDSKRATVALRWKGIDLRGVSFDLLLAAYLLNPGQDASDVAAV |
| | | AKMKQYEAVRSDEAVYGKGAKRSVPDEPALAEHLVRKAAAIW |
| | | ALERPMMDDLRRNEQDELLMALEQPLAAILADMEFTGVKVDTE |
| | | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRPDTKKVHTIFNQALTQTGRLSSTEPNLQNIPIRLEE |
| | | GRKIRQAFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPDITSRNFNVRSFAERTAINTPIQGSAADIIKKAMIDL |
| | | SARLKEERLQARLLLQVHDELILEAPSDEIERLCRIVPEVMEQAV |
| | | TLRVPLKVDYHYGPTWYDAK |
| 9 | Bst496 | MHHHHHHHHHHLAHIDFTMAECVTEEMLADKAALVVEVVEDN |
| | A639T; | YHDAPIVGIAVVSERGRFFLPSEVALADPQFRAWLADETKKKSM |
| | M792I, | FDSKRATVALRWKGIDLRGVSFDLLLAAYLLNPGQDASDVAAV |
| | “Bst496 | AKMKQYEAVRSDEAVYGKGAKRSVPDEPALAEHLVRKAAAIW |
| | ATMI” | ALERPMMDDLRRNEQDELLMALEQPLAAILADMEFTGVKVDTE |
| | | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRPDTKKVHTIFNQALTQTGRLSSTEPNLQNIPIRLEE |
| | | GRKIRQTFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPDITSRNFNVRSFAERTAINTPIQGSAADIIKKAMIDL |
| | | SARLKEERLQARLLLQVHDELILEAPSDEIERLCRIVPEVMEQAV |
| | | TLRVPLKVDYHYGPTWYDAK |
| 10 | Bst496 | MHHHHHHHHHHLAHIDFTMAECVTEEMLADKAALVVEVVEDN |
| | A639T; | YHDAPIVGIAVVSERGRFFLPSEVALADPQFRAWLADETKKKSM |
| | D775Q; | FDSKRATVALRWKGIDLRGVSFDLLLAAYLLNPGQDASDVAAV |
| | M792I, | AKMKQYEAVRSDEAVYGKGAKRSVPDEPALAEHLVRKAAAIW |
| | “Bst496 | ALERPMMDDLRRNEQDELLMALEQPLAAILADMEFTGVKVDTE |
| | TQI” | RLNQMGQELAEQLRAAERRIYELAGQEFNINSPKQLGVILFEKLQ |
| | | LPVLKKTKTGYSTSADVLEKLAPHHEIVENILHYRQLGKLQSTY |
| | | VEGLLKVVRPDTKKVHTIFNQALTQTGRLSSTEPNLQNIPIRLEE |
| | | GRKIRQTFVPSEPGWLMFAADYSQIELRVLAHIAEDDNLIEAFRR |
| | | DLDIHTKTAMDIFHVKEEDVTPNMRRQAKAVNFGIVYGISDYGL |
| | | AQNLNITRKEAAEFIERYFASFPGVKRYMETIVQEAKQKGYVTT |
| | | LLHRRRYLPQITSRNFNVRSFAERTAINTPIQGSAADIIKKAMIDL |
| | | SARLKEERLQARLLLQVHDELILEAPSDEIERLCRIVPEVMEQAV |
| | | TLRVPLKVDYHYGPTWYDAK |

Throughout the disclosure, reference is made to specific amino acid positions by identifying the position of the amino acid within a reference sequence. While either the position numbering of SEQ ID NO: 1 or SEQ ID NO: 4 could be used, position numbering relative to SEQ ID NO: 1 is used throughout the disclosure for consistency. The amino acid mutations are the same in either sequence; only the position numbers differ (due to the deletion of the 5′ to 3′ exonuclease domain from and addition of the N-terminal ten-histidine tag to SEQ ID NO: 1 to produce SEQ ID NO: 4).

In some embodiments, mutations are made in one or more (e.g., one, two, or three) amino acid positions selected from, for example, P597R, A639T, D775Q, and M792I, according to the numbering as shown in SEQ ID NO: 1.

In some embodiments, the one or more mutations is made in the amino acid position P597, according to the numbering as shown in SEQ ID NO: 1. In some embodiments, the one or more mutations is made in the amino acid position A639, according to the numbering as shown in SEQ ID NO: 1. In some embodiments, the one or more mutations is made in the amino acid position D775, according to the numbering as shown in SEQ ID NO: 1. In some embodiments, the one or more mutations is made in the amino acid position M792, according to the numbering as shown in SEQ ID NO: 1.

In some embodiments, the one or more mutations comprise one or more amino acid substitution(s) as shown in Table 1. In some embodiments, the one or more mutations comprise one or more amino acid substitution(s) selected from: P597R, A639T, D775Q, and M792I, according to the numbering as shown in SEQ ID NO: 1. In some embodiments, the one or more mutations are one or more amino acid substitution(s) selected from the group consisting of: P597R, A639T, D775Q, and M792I, according to the numbering as shown in SEQ ID NO: 1.

In some embodiments, the one or more mutations is P597R, according to the numbering as shown in SEQ ID NO: 1. In some embodiments, the one or more mutations is A639T, according to the numbering as shown in SEQ ID NO: 1. In some embodiments, the one or more mutations is D775Q, according to the numbering as shown in SEQ ID NO: 1. In some embodiments, the one or more mutations is M792I, according to the numbering as shown in SEQ ID NO: 1.

In some embodiments, the one or more mutations comprise one or more amino acid substitution(s) selected from: A639T and M792I; and A639T, D775Q, and M792I according to the numbering as shown in SEQ ID NO: 1. In some embodiments, the one or more mutations are one or more amino acid substitution(s) selected from the group consisting of: A639T and M792I; and A639T, D775Q, and M792I, according to the numbering as shown in SEQ ID NO: 1.

In some embodiments, the one or more mutations are A639T and M792I, according to the numbering as shown in SEQ ID NO: 1. In some embodiments, the one or more mutations are A639T, D775Q, and M792I, according to the numbering as shown in SEQ ID NO: 1.

In some embodiments, a Bst polymerase variant of the disclosure has an amino acid sequence as shown in Table 1. In some embodiments, a Bst polymerase variant of the disclosure comprises a polypeptide having at least 90% (e.g., at least 90%, at least 91%, at least 92%, at least 93%, at least 94%, at least 95%, at least 96%, at least 97%, at least 98%, at least 99%, or about 100%; 90-91%, 91-92%, 92-93%, 93-94%, 94-95%, 95-96%, 95-97%, 96-97%, 96-98%, 97-98%, 97-99%, 98-99%, 98-100%, or 99-100%; 90%, 91%, 92%, 93%, 94%, 95%, 96%, 97%, 98%, 99%, or 100%) sequence identity to an amino acid sequence as shown in any of SEQ ID NOs: 5-10. In some embodiments, a Bst polymerase variant of the disclosure has an amino acid sequence as shown in any one of SEQ ID NOs: 5-10.

In some embodiments, a Bst polymerase variant of the disclosure has an amino acid sequence as shown in SEQ ID NO: 4 (or an amino acid sequence having at least 90%, at least 95%, or at least 99% sequence identity thereto). In some embodiments, a Bst polymerase variant of the disclosure has an amino acid sequence as shown in SEQ ID NO: 5 (or an amino acid sequence having at least 90%, at least 95%, or at least 99% sequence identity thereto). In some embodiments, a Bst polymerase variant of the disclosure has an amino acid sequence as shown in SEQ ID NO: 6 (or an amino acid sequence having at least 90%, at least 95%, or at least 99% sequence identity thereto). In some embodiments, a Bst polymerase variant of the disclosure has an amino acid sequence as shown in SEQ ID NO: 7 (or an amino acid sequence having at least 90%, at least 95%, or at least 99% sequence identity thereto). In some embodiments, a Bst polymerase variant of the disclosure has an amino acid sequence as shown in SEQ ID NO: 8 (or an amino acid sequence having at least 90%, at least 95%, or at least 99% sequence identity thereto). In some embodiments, a Bst polymerase variant of the disclosure has an amino acid sequence as shown in SEQ ID NO: 9 (or an amino acid sequence having at least 90%, at least 95%, or at least 99% sequence identity thereto). In some embodiments, a Bst polymerase variant of the disclosure has an amino acid sequence as shown in SEQ ID NO: 10 (or an amino acid sequence having at least 90%, at least 95%, or at least 99% sequence identity thereto).

The present disclosure further provides nucleic acid sequences encoding the variant Bst polymerases described herein. In some embodiments, the nucleic acid has a sequence selected from SEQ ID Nos: 51-56.

### Isothermal Amplification

Aspects of the disclosure relate to Bst DNAP and its variants suitable for use in an amplification method, for example, an isothermal amplification method, which amplifies nucleic acid sequence(s) of interest by incubating reaction mix at a constant temperature for a certain amount of time.

In some embodiments, the Bst DNAP and its variants disclosed herein find use in LAMP or RT-LAMP for amplification of DNA and RNA. As described by Notomi et al., (2000), LAMP employs a set of primers, F3 primer, B3 primer, FIP, and BIP, specific for amplification of target nucleic acid (e.g., DNA or RNA). In addition, two more primers, Forward loop (FL) and Backword loop (BL), can be included in the reaction mix to speed up the time to results (in minutes) and improve LAMP assay performance.

In some embodiments, LAMP primers specific for a given target nucleic acid (DNA/RNA) can be designed using software e.g., [PrimerExplorer (primerexplorer.jp/e); NEB LAMP primer design tool (lamp.neb.com/#!/); LAVA (Torres et al, 2011); LAMP Designer (www.premierbiosoft.com/isothermal/lamp.html)].

In some embodiments, LAMP is an isothermal method of amplification that involves running a reaction at a constant temperature ranging from 60 to 72° C. for a duration of time, often ranging from 15 minutes to 1 hour (Notomi et al., 2000).

In some embodiments, LAMP involves use of Bst DNAP with strand displacement activity but lacks 5′-3′ exonuclease activity. The Bst DNAP may be a wild-type or naturally occurring polymerase, or may be a commercially available polymerase. For amplification of RNA targets by LAMP (RT-LAMP), a secondary enzyme, RT polymerase, is included in the reaction mix. In some embodiments, the Bst DNAP is selected from, for example, the wild-type Bst496 DNAP with a ten-histidine tag at its N-terminus but lacking 5′-3′ exonuclease activity (WT-Δexo-Bst496; SEQ ID NO: 4), Bst 2.0 WarmStart (New England Biolabs, Cat. No. M0357), or Bst 3.0 (New England Biolabs, Cat. No. M0374).

Bst DNAPs are primarily used for amplification of DNA targets because of their DNA-dependent DNA polymerase activity. Also, most Bst DNAPs have rudimentary reverse transcriptase (RT) activity but it is not enough to efficiently amplify RNA targets. Therefore, an RT polymerase is usually used in LAMP along with a DNAP for amplification of RNA targets (RT-LAMP). However, as described herein, the present disclosure provides Bst DNAP with increased RT activity that is strong enough to be used as a standalone enzyme in a RT-LAMP reaction. The Bst DNAP variants described herein can thus act as DNA polymerases and/or as reverse transcriptases, depending on the identity of the target nucleic acid (DNA or RNA) without any changes in the reaction conditions or use of additional enzyme (RT polymerase).

Enzymes having reverse transcriptase activity (e.g., “reverse transcriptases”) are described in, for example, Kati et al., (1992); J. Biol. Chem., 267(36): 25988-97; Kotewicz et al., (1985); and Gene, 35(3): 249-58); and include, for example, WarmStart® RTx (New England Biolabs, Cat. No. M0380); SuperScript IV (ThermoFisher Scientific, Cat. No. 18090010); and M-MLV (ThermoFisher Scientific, Cat. No. 28025013). Any suitable reverse transcriptase may be used as the second enzyme having reverse transcriptase activity. In some embodiments, the second enzyme having reverse transcriptase activity is WarmStart® RTx Reverse Transcriptase (New England Biolabs, Cat. No. M0380) or a Human Immunodeficiency Virus (HIV) reverse transcriptase (Thunder™ RT polymerase, Cat. No. 7100; Varizymes, Middleton, WI).

In some embodiments, an amplification reaction mixture (e.g., a LAMP reaction mixture) comprises one or more LAMP primers and one or more additional reagents. In some embodiments, one or more (and, in some instances, each) additional reagents are in liquid form (e.g., in solution). In some embodiments, one or more (and, in some instances, each) additional reagents are in solid form (e.g., lyophilized, dried, crystallized, air jetted).

In certain embodiments, the one or more additional reagents comprise one or more lysis reagents. A lysis reagent generally refers to a reagent that promotes cell lysis either alone or in combination with one or more reagents and/or conditions (e.g., heating). In some cases, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the one or more lysis reagents comprise an RNase inhibitor (e.g., a murine RNase inhibitor).

Aspects of the disclosure relate to methods of detecting a target nucleic acid sequence using the Bst polymerase variants of the disclosure. In some embodiments, the method comprises: a) obtaining a biological sample from a subject; b) performing a nucleic acid amplification reaction configured to amplify the target nucleic acid sequence using a Bst polymerase variant of the disclosure; and c) detecting the presence or absence of the target nucleic acid sequence. In some embodiments, the method further comprises a step of adding a second enzyme having reverse transcriptase activity to the nucleic acid amplification reaction. Such second enzymes having reverse transcriptase activity are described elsewhere herein.

In some embodiments, components for performing an assay (e.g., LAMP assay) are provided in the form of a kit. In some embodiments, kits comprise variant Bst polymerases described herein, along with any additional reagents useful, sufficient, or necessary for performing an assay. Examples include but are not limited to: primers, probes, RT enzymes, buffers, controls, and the like.

Instructions for using the kits to perform one or more methods of the disclosure can be provided and can be provided in any fixed medium. The instructions may be located inside or outside a container or housing, and/or may be printed on the interior or exterior of any surface thereof. A kit may be in multiplex form for concurrently detecting and/or quantitating one or more different target polynucleotides.

In some embodiments, the target nucleic acid sequence is a DNA sequence or an RNA sequence. In some embodiments, nucleic acid amplification reaction comprises LAMP or RT-LAMP. In some embodiments, wherein the target nucleic acid sequence is a DNA sequence, the nucleic acid amplification reaction comprises LAMP. In some embodiments, wherein the target nucleic acid sequence is an RNA sequence, the nucleic acid amplification reaction comprises RT-LAMP.

In some embodiments, a method of detection comprises obtaining a biological sample from a subject. Examples of biological samples include bodily fluids (e.g., mucus, saliva, blood, serum, plasma, amniotic fluid, sputum, urine, cerebrospinal fluid, lymph, tear fluid, feces, gastric fluid, vaginal fluid, or semen), cell scrapings (e.g., a scraping from the mouth or interior check), exhaled breath particles, or tissue extracts. In some embodiments, the biological sample comprises a mucus, saliva, sputum, blood, urine, vaginal, semen, or cell scraping sample.

In some embodiments, amplified nucleic acid sequences (i.e., amplicons) may be detected using any suitable method. In some embodiments, a target nucleic acid is detected using a lateral flow assay (LFA) strip, a colorimetric assay, a CRISPR/Cas method of detection, or is directly detected using hybridization.

To facilitate detection of a target nucleic acid, in some embodiments, one or more LAMP primers may be chemically modified. In some embodiments, such chemical modification comprises the conjugation of one or more LAMP primers to a detectable label. In certain embodiments, the detectable label is a fluorescent label. In some instances, the fluorescent label is associated with a quenching moiety that prevents the fluorescent label from signaling until the quenching moiety is removed. Conjugation of one or more LAMP primers to a detectable label may be desirable in certain embodiments to visualize readout results, for example on a lateral flow assay strip. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay, as described elsewhere herein).

Devices useful for performing methods of the disclosure are also provided. The devices can comprise means for detecting the presence of a target molecule, for example components for performing one or more methods of nucleic acid extraction, amplification, and/or detection. Such components may include one or more of an amplification chamber (for example, a thermal cycler), a plate reader, a spectrophotometer, capillary electrophoresis apparatus, a chip reader, and or robotic sample handling components. These components ultimately can obtain data that reflects the presence of the target molecules used in the assay being employed.

The variant Bst polymerases described herein may be used in any application utilizing a strand-displacing polymerase. In some embodiments, a Bst polymerase variant described herein is used in a nucleic acid sequencing method. In certain embodiments, the nucleic acid sequencing method is a long-read sequencing method. In certain embodiments, the nucleic acid sequencing method is a short-read sequencing method. In some embodiments, the nucleic acid sequencing method is a next-generation sequencing method.

### Experimental

### Example 1

### Development of Variant Bst496 DNA Polymerases

Nucleic acid polymerases comprising one or more mutations relative to a full-length, wild-type Bacillus stearothermophilus (Bst) nucleic acid polymerase were designed. As described elsewhere herein, the full-length, wild-type Bst sequence used is that of Geobacillus sp. C56-T2 (Genbank Accession No. TWG31496.1, SEQ ID NO: 1), termed “FL-WT-Bst496”.

The Bst polymerases described herein have both DNA-dependent DNA polymerase and reverse transcriptase (RT) capabilities and can amplify both DNA and RNA targets without the addition of a second enzyme having RT activity.

### Optimization of Wild-Type Bst496 DNAP Sequence

The full-length, wild-type Bst496 nucleic acid polymerase (SEQ ID NO: 1) was optimized for LAMP by removal of the 5′ to 3′ exonuclease domain (SEQ ID NO: 2) and for purification by addition of an N-terminal ten-histidine tag (10XHis, SEQ ID NO: 3).

First, the 5′ to 3′ exonuclease domain of full-length, wild-type Bst496 (Geobacillus sp. C56-T2 (Genbank Accession No. TWG31496.1); SEQ ID NO: 1), located at the N-terminus of the wild-type protein, was deleted. The 5′ to 3′ exonuclease domain which was deleted is shown as SEQ ID NO: 2. This deletion was made to avoid nucleolytic release of any fluorophores and quenchers conjugated to the LAMP primers.

Second, a ten-histidine tag was added to the N-terminus of the 5′ to 3′ exonuclease domain-deficient Bst496. The ten-histidine tag which was added is shown as SEQ ID NO: 3. This addition was made to enable traditional Ni-affinity column protein purification.

The resultant Bst496 sequence which comprises a deletion of a 5′ to 3′ exonuclease domain (SEQ ID NO: 2) and an addition of an N-terminal ten-histidine tag (SEQ ID NO: 3) is shown as SEQ ID NO: 4 and is referred to herein as “WT-Δexo-Bst496.”

### Generation of Bst496 DNAP Variants

The Bst polymerase variants of the disclosure were designed to comprise one or more mutations relative to either the full-length, wild-type Bst496 sequence (Geobacillus sp. C56-T2 (Genbank Accession No. TWG31496.1)) shown in SEQ ID NO: 1 or to the WT-Δexo-Bst496 sequence shown in SEQ ID NO: 4. The mutations are shown in Table 1. As described elsewhere herein, any of the mutations shown in Table I may be made alone or in combination. However, specific combinations of mutations which have demonstrated high reverse transcriptase activity (see Example 3) and/or DNA-dependent DNA polymerase activity (see Example 3) are noted in Table 1 as SEQ ID NOs: 5-10.

For clarity, position numbering relative to SEQ ID NO: 1 (FL-WT-Bst496) is used throughout the application. As will be understood, the amino acid mutation(s) made are the same in SEQ ID NO: 4 (WT-Δexo-Bst496); only the position number will differ (due to the deletion of the 5′ to 3′ exonuclease domain from and addition of the N-terminal ten-histidine tag to SEQ ID NO: 1 to produce SEQ ID NO: 4).

The resultant Bst polymerase variants comprising one or more mutations—relative to either the full-length, wild-type Bst496 sequence (Geobacillus sp. C56-T2 (Genbank Accession No. TWG31496.1)) shown in SEQ ID NO: 1 or to the WT-Δexo-Bst496 sequence shown in SEQ ID NO: 4—are shown as SEQ ID NOs: 5-10.

### Method of Production

The Bst polymerase variants of the disclosure were manufactured and purified as follows. The initial plasmid of SEQ ID NO: 4 was ordered synthesized by ATUM (Newark, CA). All point mutations were introduced using Agilent's QuikChange II XL site-directed mutagenesis kit (Cat No. 200522) and the primers in Table 2 (SEQ ID NOs: 11-18), then subcloned into and expressed in Lucigen's E. cloni 10G cell line (60108-1). Isolates of E. cloni 10G containing plasmids encoding Bst496 variants were stored in 50% glycerol at −80° C. before being grown in 100 mL Luria Broth (LB) with 30 μg/mL kanamycin and 0.4% glucose at 200 rpm and 30° C. overnight. The culture was then transferred to 2 L LB with 30 μg/mL kanamycin and 0.4% rhamnose for induction of protein expression for 24 hrs at 200 rpm and 30° C. The culture was centrifuged at 2200 rcf for 20 minutes and the pellet resuspended in 100 mM Tris-HCl pH 8.0with 500 mM NaCl and 30 mM imidazole at a ratio of 10 mL per gram of wet cell paste. The cell suspension was sonicated using a Branson digital sonifier for 9 minutes using 15 s pulses at 50% power and 0° C. After sonication, the lysate was centrifuged for 30 minutes at 4° C. and 11000 rcf to clarify. The clarified lysate was applied to ThermoFisher HisPur Ni column equilibrated with 100 mM Tris-HCl pH 8.0 with 500 mM NaCl and 30 mM imidazole. The column was washed with 10 column volumes of 100 mM Tris-HCl pH 8.0 with 500 mM NaCl and 30 mM imidazole, 10 column volumes of 100 mM Tris-HCl pH 8.0 with 150 mM NaCl and 30 mM imidazole. The protein was eluted with 5 column volumes 100 mM Tris-HCl pH 8.0 with 100 mM NaCl and 300 mM Imidazole. The protein elution UV absorbance peak was collected and applied to a Q-sepharose column normalized with 100 mM Tris-HCl pH 8.0 with 150 mM NaCl. The protein does not bind to the Q-sepharose column; therefore, the flow-through UV absorbance peak was collected.

Fractions from Q-Sepharose containing the enzyme were pooled and concentrated to 10-20 mL by ultrafiltration using a Pierce Protein Concentrator with a 10 kDa molecular weight cutoff (ThermoFisher Scientific, Cat. No. 88527). The concentrate was dialyzed against 50 volumes of storage buffer (50 mM Tris-HCl pH 7.5 with 50 mM KCl, 1 mM DTT, 0.1 mM EDTA, 0.05% Tween-20, 0.05% Nonidet-P40 substitute, and 50% glycerol).

### Reverse Transcriptase Activity of Bst496 DNAP and its Variants

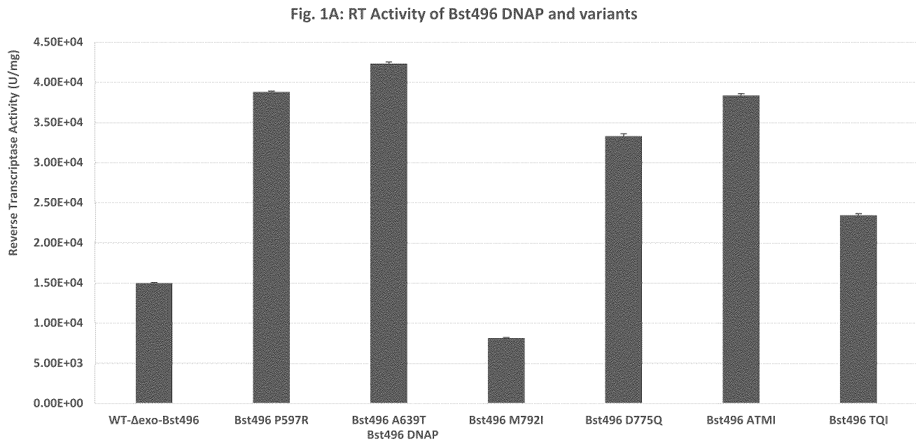

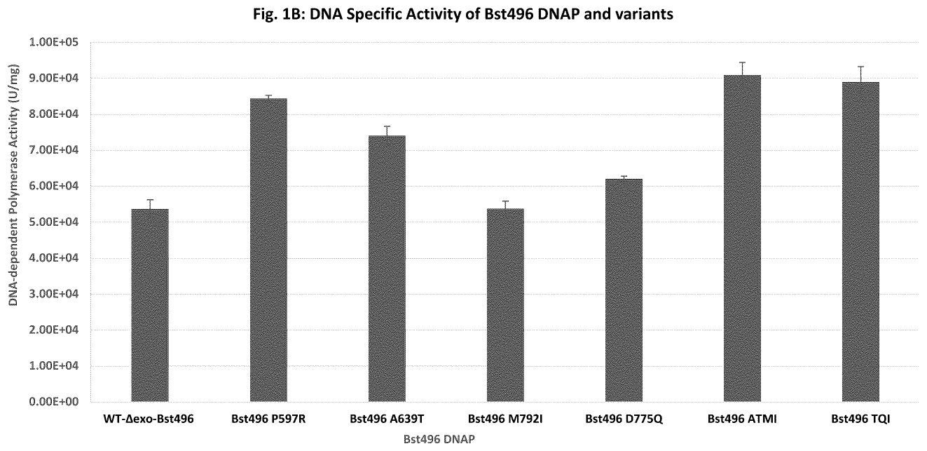

Reverse transcriptase activity of Bst496 DNAP and its variants was quantified using a modified SYBR Green I (ThermoFisher Scientific, Cat. No. S7563), Product-Enhanced Reverse Transcriptase PCR assay (SG-PERT; Vermeire et al. (2012), PLOS ONE, 7(12): e50859), using 12.5 ng of MS2 RNA in a 25 μL reaction and Q5 High-fidelity HotStart (New England Biolabs, Cat. No. M0493) as the DNA polymerase. This system was customized to use PicoGreen (ThermoFisher Scientific, Cat. No. P7581) instead of SYBR Green (PG-PERT), primers MS2 F3b/R, and Q5 HotStart High-fidelity DNA polymerase. The following 1× reaction recipe was used: 20.0 μL H₂O, 2.5 μL 10× Isothermal amplification buffer (New England Biolabs, Cat. No. B0537) for Bst 3.0 or 10× Isothermal buffer (Varizymes; 20 mM Tris-HCl [pH 8.3], 10 mM K₂SO₄, 50 mM KCl, 2 mM MgSO₄, 0.1% Tween-20) for WT-Δmexo-Bst496 DNAP and its variants, 1 μL PicoGreen 1:16 diluted in H₂O, 0.5 μL 10 mM ca. dNTPs (New England Biolabs, Cat. No. N0447), 0.5 μL murine RNase inhibitor (New England Biolabs, Cat. No. M0314), 0.25 μL Q5 Hot Start High-Fidelity DNA polymerase, 0.125 μL 100 μM MS2 F3b/R (IDT, SEQ ID NOs 44-45, Table 3), and 0.125 μL 100 ng/μL untreated MS2 phage (Varizymes, Middleton, WI). The enzyme dilution range was 6.4 pg to 100 ng for Bst496 variants and 0.5 mU to 8 U for Bst 3.0, each in separate 25 μL reactions. The following thermocycler program was used: 1) 61.5° C., 20 minutes; 2) 98° C., 3 minutes; 3) 98° C., 5 seconds; 4) 56° C., 15 seconds; 5 ) 72° C., 15seconds, plate read on SYBR channel; 6) go to step 3, 39×. Results were visualized with 1× PicoGreen dye. A standard curve of Bst 3.0 (New England Biolabs, Cat. No. M0374) was made using the C_{q}s of a triplicate, 7-step, 5-fold serial dilution from 8 U per 25 μL reaction. The RT activity of each enzyme was determined relative to known amounts of Bst 3.0, and the results of FIG. 1A are shown in Units of Bst 3.0 activity (U)/mg. All Bst496 variants retained RT activity, with Bst496 P597R, Bst496 A639T, and Bst496 ATMI showing 2.3-fold the amount of activity compared to WT-Δexo-Bst496.

### DNA-Dependent Polymerase Activity of Bst496 DNAP and its Variants

DNA-dependent polymerase activity for WT-Δexo-Bst496 DNAP and variants thereof was assessed using a single-stranded M13 primer extension assay with SYTO9 (ThermoFisher, Cat. No. S34854), with Bst 2.0 WarmStart (New England Biolabs, Cat. No. M0538) as a standard. The following 1× reaction recipe was used: 12.65 μL H₂O, 2 μmL 10× Isothermal amplification buffer (New England Biolabs, Cat. No. B0537) for Bst 2.0 WarmStart or 10× Isothermal buffer (Varizymes; 20 mM Tris-HCl [pH 8.3], 10 mM K₂SO₄, 50 mM KCl, 2 mM MgSO₄, 0.1% Tween-20) for WT-Δexo-Bst496 DNAP and its variants, 1.2 μL 100 mM MgSO4 (New England Biolabs, Cat. No. B1003), 1.4 μL 25 mM ca. dNTPs, 0.75 μL 100 μM SYTO9, 1 μL 1 μg/μL ssM13mp18 (Bayou Biolabs, Cat. No. P-107), 1 μL 10 μM M13FT-41 (IDT, SEQ ID NO: 46, Table 3). Reactions were incubated in a CFX-96 thermocycler at 61.5° C. for 40 cycles, with each cycle being a SYBR plate read every 3 seconds. A standard curve of Bst 2.0 WS was made using the initial slopes of the amplification curves of a triplicate, 7-step, 2-fold serial dilution from 1 U per 25 μL reaction. The activities of the Bst496 variants described herein were calculated from linear, in-range measurements derived from a similar dilution from 30 ng of polymerase/25 μL reaction. The DNA-dependent DNA polymerase activity of each enzyme was calculated relative to known amounts of Bst 2.0 WS, and the results of FIG. 1B are shown in Units of Bst 2.0 WS activity (U/mg).

The following Bst496 DNAPs were assessed: WT-Δexo-Bst496 (SEQ ID NO: 4), Bst496 P597R (SEQ ID NO: 5), Bst496 A639T (SEQ ID NO: 6), Bst496 D775Q (SEQ ID NO: 7), Bst496 M792I (SEQ ID NO: 8), Bst496 ATMI (SEQ ID NO: 9), and Bst496 TQI (SEQ ID NO: 10). As shown in FIG. 1B, all Bst496 variants possess DNA-dependent polymerase activity. Bst496 M792I had the same DNA-dependent polymerase activity as the wildtype, while all other variants had enhanced DNAP activity.

### Performance of Bs1496 DNAP and its Variants in LAMP Assays

Performance of Bst496 DNAP and variants thereof was evaluated in LAMP assays to amplify multiple RNA and DNA targets. Ability of Bst496 DNAP variants to amplify RNA and DNA targets was assessed relative to the WT-Δexo-Bst496 sequence shown in SEQ ID NO: 4, Bst 2.0 WS (New England Biolabs, Cat. No. M0537), and Bst 3.0 (New England Biolabs, Cat. No. M0374).

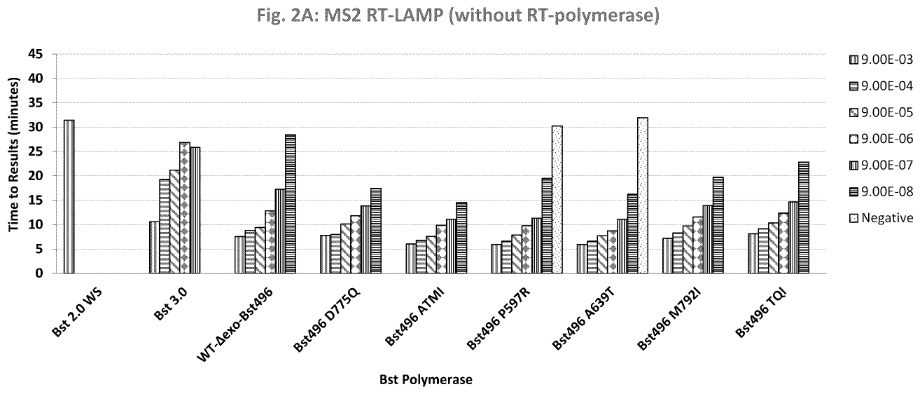

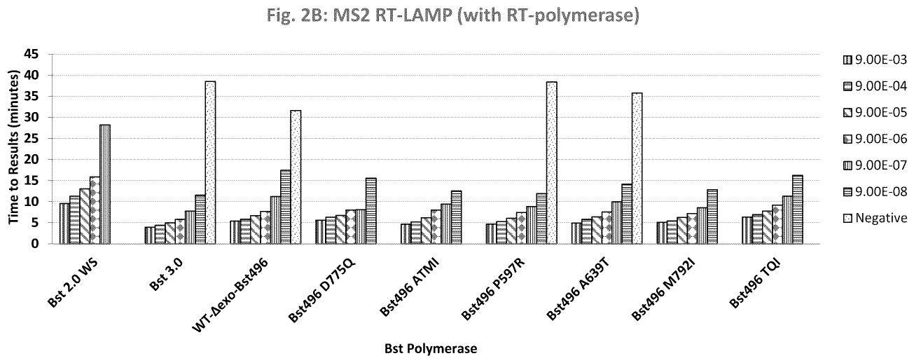

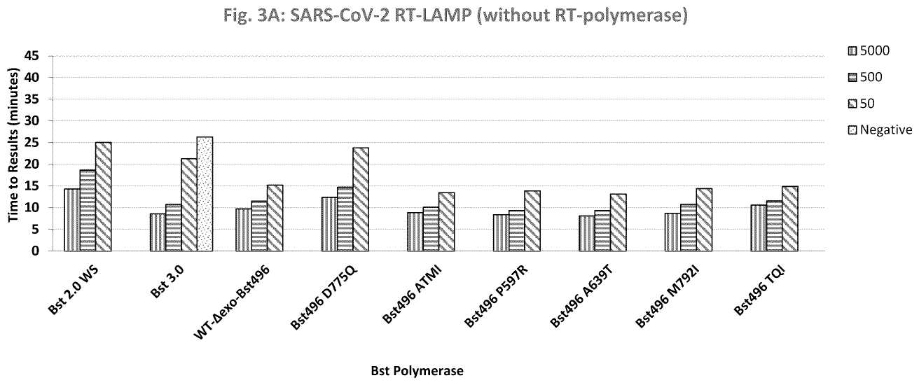

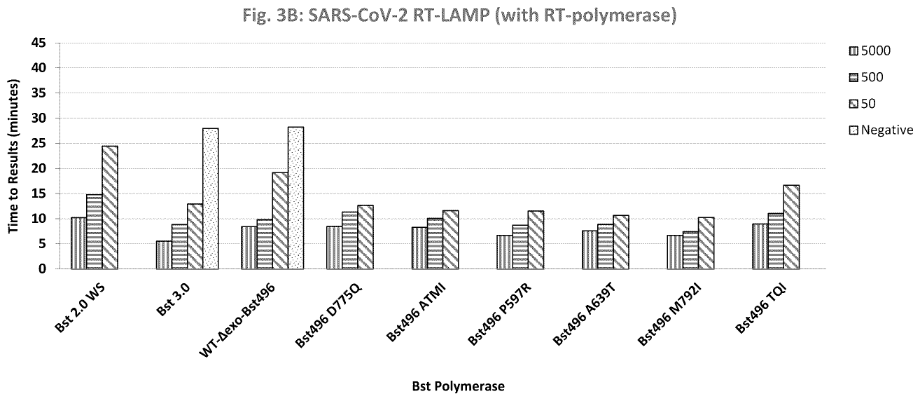

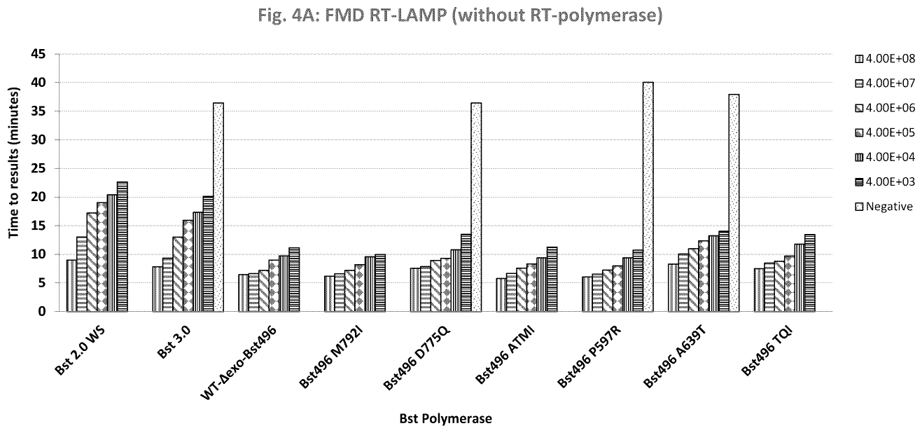

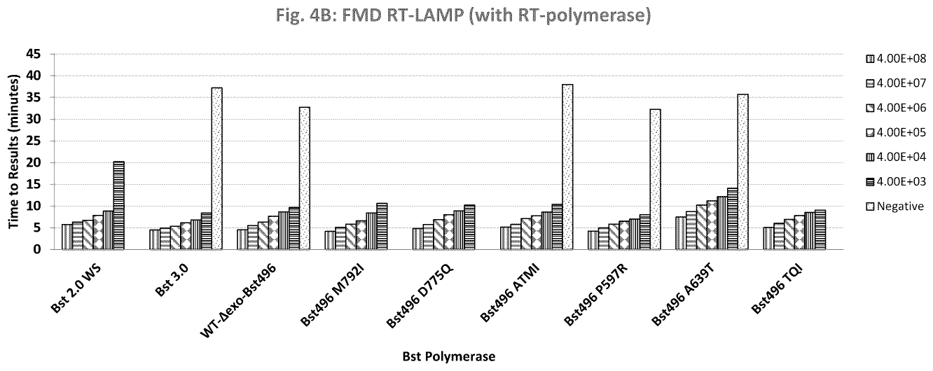

The variants of Bst496 DNAP described herein showed enhanced RT polymerase activity and amplified different RNA targets more efficiently in LAMP reactions at a wide temperature range (64° C.-72° C.) without addition of a second polymerase (RT polymerase) as compared to WT-Δexo-Bst496 (SEQ ID NO: 4), and existing state-of-the-art Bst polymerases, Bst 2.0 WS and Bst 3.0 (both from New England Biolabs, MA). Even after addition of a polymerase with RT activity (RT polymerase), LAMP reactions with Bst496 variants yielded superior results as compared WT-Δexo-Bst496 (SEQ ID NO: 4), and existing state-of-the-art Bst polymerases, Bst 2.0 WS and Bst 3.0 (both from New England Biolabs, MA). Results are shown in FIG. 2A and 2B, FIG. 3A and 3B, and FIG. 4A and 4B.

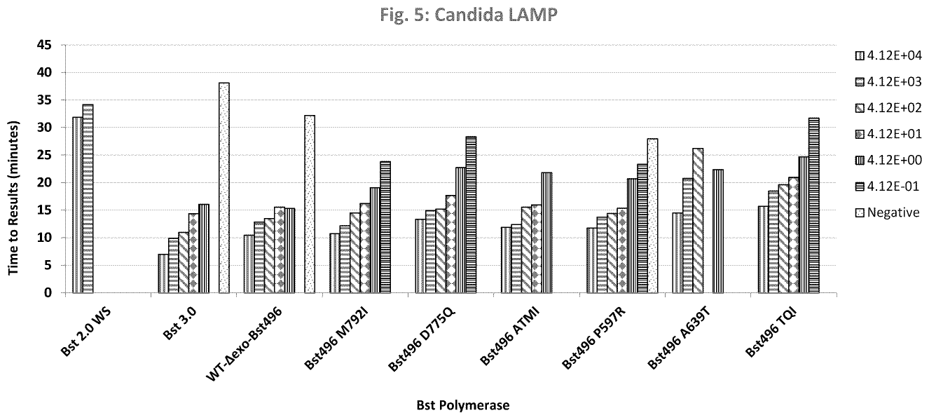

Similar results were obtained in a LAMP reaction for amplification of DNA targets. In DNA LAMP assays, faster time to results (in minutes) was achieved with Bst496 DNAP variants as compared to WT-Δexo-Bst496 (SEQ ID NO: 4), and existing state-of-the-art Bst polymerases, Bst 2.0 WS and Bst 3.0 (both from New England Biolabs, MA) indicating strong DNA dependent DNA polymerase activity of Bst496 DNAP variants in addition to RT activity. Results are presented in FIG. 5.

### Evaluation of RT-Activity of Bst496 DNAP in LAMP Assays

Bst496 DNAP variants as described herein exhibited strong RT activity sufficient for amplification of RNA targets without the use of an additional enzyme (RT). This is significant because, typically for amplification of RNA targets, a secondary enzyme with RT activity (RT) such as AMV or M-MLV, in addition to DNA polymerase (such as Bst polymerase or Taq polymerase) is included in the reaction mix for optimal amplification of RNA targets. This use of additional enzyme (RT polymerase) creates issues with reaction conditions optimization as well as adds to the overall cost of the reaction. However, in certain embodiments, addition of RT to the reaction mix containing Bst496 DNAP improved the performance (faster time to results) of RNA amplification in a LAMP reaction.

The RT activity of Bst496 polymerase variants described herein was evaluated in RT-LAMP reactions for different RNA targets, both with and without additional RT. Results were compared to commercially available Bst polymerases (Bst 2.0 WarmStart and Bst 3.0; New England Biolabs, MA). Results are presented in FIGS. 2-5.

WT-Δexo-Bst496 DNAP and its variants: Bst496 P597R (SEQ ID NO: 5), Bst496 A639T (SEQ ID NO: 6), Bst496 D775Q (SEQ ID NO: 7), Bst496 M792I (SEQ ID NO: 8), Bst496 ATMI (SEQ ID NO: 9), and Bst496 TQI (SEQ ID NO: 10) were used in a LAMP reaction to amplify different RNA targets (MS2, FMDV, and SARS-COV-2) both with and without the addition of a secondary reverse transcriptase enzyme. Performance of WT-Δexo-Bst496 DNAP and its variants was compared with Bst 2.0 WarmStart and Bst 3.0, both with and without the addition of a secondary enzyme (RT). For a given RNA target, LAMP reactions with different polymerases (WT-Δexo-Bst496; Bst496 variants; Bst 2.0 WarmStart and Bst 3.0) were carried out at the same temperature and under similar conditions on the same instrument (CFX-96, BioRad). However, different reaction buffers, depending on the Bst DNAP, were used. All LAMP reactions with WT-Δexo-Bst496 DNAP and its variants were carried out using 10× Isothermal buffer (Varizymes, Middleton, WI). For Bst 2.0 WarmStart and Bst 3.0, 10× IsoAmp I Buffer I (New England Biolabs, MA) and IsoAmp II Buffer (New England Biolabs, MA), respectively, were used. Bar graphs shown in FIGS. 3-5 depict time to results (TTR) in minutes for each polymerase used. Short bars are considered better as it means faster TTR which in turn is the measure of amplification efficiency of a given Bst DNAP at a given temperature.

Briefly, the method involved preparing 10-fold serial dilutions of RNA in appropriate diluent such as water and adding each dilution to a reaction mix containing optimized concentrations of MgSO₄, dNTP mix, Bst DNAP, target-specific primers, and intercalating dye (SYTO-82) for real-time monitoring of nucleic acid amplification. The reactions were performed in a real-time thermocycler (CFX-96, BioRad) for 60 cycles with each cycle being 30 seconds at a temperature optimum for each target. SARS-COV-2 LAMP reactions were carried out at 64° C., FMDV LAMP reactions were carried out at 68° C., and MS2 LAMP reactions were carried out at 72° C. Reaction kinetics were monitored in real-time by measuring the increase in fluorescence in the HEX channel associated with the accumulation of double-stranded DNA by LAMP.

As shown in FIGS. 3-5, Bst496 DNAP variants showed superior performance (in terms of sensitivity and TTR) in amplifying the target RNA without addition of RT, as compared to WT-Δexo-Bst496, Bst 2.0 WarmStart, and Bst 3.0. Similar results were obtained in the LAMP reaction where a secondary enzyme (RT) was added to the reaction mix in addition to Bst DNAP.

In MS2 LAMP reactions (without additional RT), the limit of detection (LOD) was 9×10⁻⁸ μg/25 μL within 10-16 minutes with Bst496 variants, with the fastest TTR of 10.32 minutes with Bst496 ATMI and the slowest TTR of 16.2 minutes with Bst496 TQI (FIG. 2A). Similar LOD was obtained using WT-Δexo-Bst496 but it took much longer (20 minutes TTR). Results observed show that Bst496 variants described herein were much faster than Bst 3.0 (LOD: 9×10⁻⁷ μg/25 μL reaction in 18 minutes) and Bst 2.0 WarmStart (LOD: 9×10⁻³ μg/25 μL reaction in 22 minutes).

The addition of a secondary RT enzyme improved the TTR for all Bst DNAPs which were evaluated (Bst 2.0 WarmStart, Bst 3.0, WT-Δexo-Bst496, Bst496 variants: P597R, A639T, D775Q, M792I, ATMI, and TQI). This improvement in TTR and LOD was less noticeable with Bst496 ATMI (LOD 9×10⁻⁸ μg/25 μL reaction in 9 minutes), Bst496 D775Q (LOD 9×10⁻⁸ μg/25 μL reaction in 11 minutes), and Bst496 A639T (LOD 9×10⁻⁸ μg/25 μL reaction in 10 minutes) but it did improve the performance of LAMP reactions with WT-Δexo-Bst496 (LOD 9×10⁻⁸ μg/25 μL reaction in 12.5 minutes), Bst496 P597R (LOD 9×10⁻⁸ μg/25 μL reaction in 8.4 minutes), Bst496 M7921 (LOD 9×10⁻⁸ μg/25 μL reaction in 9.1 minutes), Bst496 TQI (LOD 9×10⁻⁸ μg/25 μL reaction in 11.5 minutes), Bst 3.0 (LOD 9×10⁻⁸ μg/25 μL reaction in 8.2 minutes), and Bst 2.0 WarmStart (LOD 9×10⁻⁷ μg/25 μL reaction in 20 minutes) (FIG. 2B).

In SARS-COV-2 LAMP reactions without added RT, the observed LOD was 50copies/25 μL in 10 minutes for Bst496 ATMI, Bst496 P597R, and Bst496 A639T; 10.5 minutes for WT-Δexo-Bst496, Bst496 M792I, and Bst496 TQI; and 17 minutes for Bst496 D775Q (FIG. 3A). In comparison, the observed LOD with Bst 2.0 WarmStart was 50 copies/25 μL in 17.7 minutes and 50 copies/25 μL in 15 minutes for Bst 3.0 (FIG. 3A). Addition of secondary enzyme with RT activity, helped in improving the TTR for Bst496 D775Q (LOD 50 copies/25 μL reaction in 9.0 minutes), Bst496 ATMI (LOD 50 copies/25 μL reaction in 8.2 minutes), Bst496 P597R (LOD 50 copies/25 μL reaction in 8.2 minutes), Bst496 A639T (LOD 50 copies/25 μL reaction in 7.5 minutes), Bst496 M7921 (LOD 50 copies/25 μL reaction in 7.2 minutes), and Bst 3.0 (LOD 50 copies/25 μL reaction in 9.1 minutes) but not for WT-Δexo-Bst496 (LOD 50 copies/25 μL reaction in 13.6 minutes), Bst496 TQI (LOD 50 copies/25 μL reaction in 11.8 minutes) and Bst 2.0 WarmStart (50 copies/25 μL reaction in 17.3 minutes) (FIG. 3B).

In FMDV LAMP reactions without added RT, the observed LOD was 4×10³ copies/20 μL reaction in 8 minutes for Bst496 ATMI, 9.6 minutes for Bst496 D775Q, 7 minutes for Bst496 M792I, and 7.9 minutes for WT-Δexo-Bst496 (FIG. 4). In comparison the observed LOD with Bst 2.0 WarmStart was 4×10³ copies/20 μL reaction in 16 minutes and with Bst 3.0 LOD was 4×10³ copies/20 μL reaction in 14.3 minutes (FIG. 4A). Addition of secondary enzyme with RT activity (RT), helped in improving the TTR for Bst496 D775Q (4×10³ copies/20 μmL reaction in 7.2 minutes), WT-Δexo-Bst496 (4×10³ copies/20 μL reaction in 7 minutes), Bst 2.0 WarmStart (4×10³ copies/20 μL reaction in 14.3 minutes), and Bst 3.0 (4×10³ copies/20 μL reaction in 6 minutes) but not for Bst496 ATMI (4×10³ copies/20 μL reaction in 7.4 minutes) and Bst496 M7921 (4×10³ copies/20 μL reaction in 7.6 minutes) (FIG. 4B).

### Evaluation of Bst496 DNAP and its Variants for Amplification of DNA Targets in LAMP Assay

Many commercially available Bst DNAPs have 5′-3′ DNA polymerase activity and strong strand displacement activity but lack 5′-3′ exonuclease activity. The strong strand displacement activity enables Bst DNAP to synthesize DNA at a constant temperature. The WT-Δexo-Bst496 DNAP and its variants as described in this disclosure maintained strong DNA polymerase activity in addition to enhanced RT activity as evident from amplification of the DNA and RNA targets. This is significant because only one enzyme, Bst496 DNAP and its variants, is needed for amplification of DNA and RNA targets. A Bst polymerase with strong DNA polymerase activity in addition to RT activity is useful for conditions where a single reaction mix for amplification of both DNA and RNA in a single LAMP reaction is desired.

The performance of WT-Δexo-Bst496 and its variants was evaluated in a DNA LAMP and compared with other commercially available Bst polymerases, Bst 2.0 WS, and Bst 3.0 (New England Biolabs, MA). WT-Δexo-Bst496 and its variants: Bst496 D775Q (SEQ ID NO: 7), Bst496 M792I (SEQ ID NO: 8), and Bst496 ATMI (SEQ ID NO: 9) were used in a LAMP reaction to amplify Candida auris genomic DNA. LAMP reactions with different polymerases (WT-Δexo-Bst496, Bst496 variants, Bst 2.0 WS, and Bst 3.0) were carried out at the same temperature and under similar conditions on same instrument (CFX-96, BioRad). However, different reaction buffers depending on the Bst DNAP, were used. LAMP reactions with WT-Δexo-Bst496 and its variants were carried out using 10× Isothermal buffer (Varizymes, Middleton, WI). For Bst 2.0 WS and Bst 3.0, 10× IsoAmp I Buffer I (New England Biolabs, MA) and IsoAmp II Buffer (New England Biolabs, MA), respectively, were used. Bar graphs shown in FIG. 5 depict time to results (TTR) in minutes for each polymerase used. Short bars are considered better as it means faster TTR which in turn is the measure of polymerase activity at a given temperature.

Briefly, the method involved preparing 10-fold serial dilutions of the target DNA and adding each dilution to a reaction mix containing optimized concentrations of MgSO₄, dNTP mix, Bst polymerase, target-specific primers, and intercalating dye (SYTO-82) for real-time monitoring of nucleic acid amplification. The reactions were performed in a real-time thermocycler (CFX-96, BioRad) for 60 cycles with each cycle being of 30 seconds at 72° C. Reaction kinetics were monitored in real-time by measuring the increase in fluorescence in HEX channel associated with the accumulation of double-stranded DNA by LAMP.

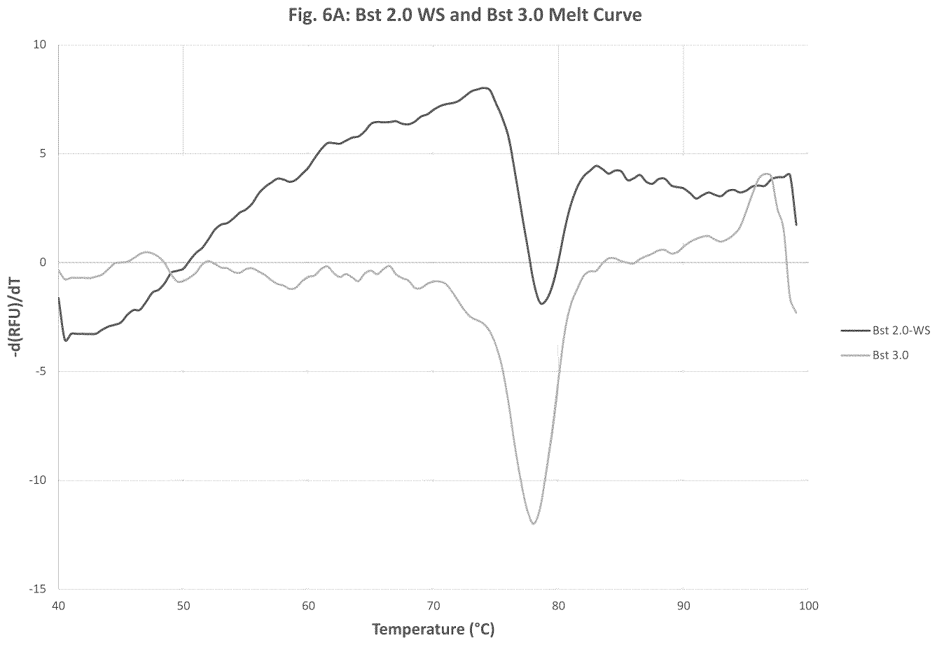

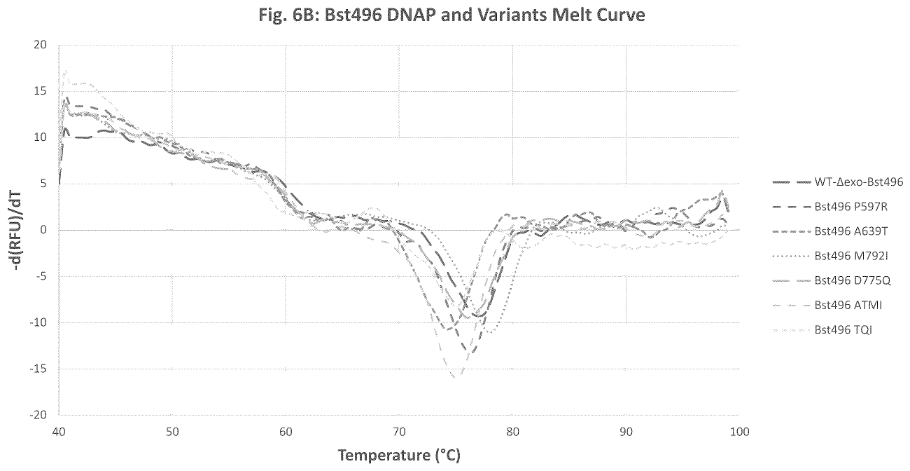

As shown in FIG. 6, Bst496 polymerase variants showed superior performance (in terms of sensitivity and TTR) in amplifying the C. auris DNA as compared to WT-Δexo-Bst496, Bst 2.0 WS, and Bst 3.0. The LOD was 0.4 pg/25 μL reaction in 20 minutes with Bst496 D775Q, 22.5 minutes with Bst496 TQI, 17 minutes with Bst496 M792I, and 16.5 minutes with Bst496 P597R (FIG. 5). As compared to this, LOD with Bst496 ATMI was 4.1 pg/25 μL reaction in 15.5 minutes, 4.1 pg/25 μL reaction in 15.9 minutes with Bst 496 A639T, 4.1 pg/25 μL reaction in 11 minutes with WT-Δexo-Bst496, 4.1 pg/25 μL reaction in 11.4 minutes with Bst 3.0, and LOD was 4120 pg/25 μL reaction in 24.2 minutes with Bst 2.0 WS (FIG. 5).

### Thermal Shift Assay

Melting temperature (Tₘ) of WT-Δexo-Bst496 DNAP and its variants was determined by running a thermal shift assay using Protein Thermal Shift Dye Kit™ (ThermoFisher; 4461146) to measure the thermal stability of the enzymes. A reaction mixture of 5 μL Protein Thermal Shift Buffer and 2.5 μL 8× Protein Thermal Shift Dye were aliquoted into a 96-well plate (unskirted, clear, Olympus Plastics #22-319NS). 12.5 μL of water containing 16 units of Bst 2.0 WarmStart/Bst 3.0 or 1 μg of WT-Δexo-Bst496 or variants was pipette-mixed into the reaction mixture. The plate was placed in the CFX-96 thermocycler and protocol was initiated. The temperature was raised 0.5° C. per second from 40° C. to 99° C., with a ROX fluorescence read for every temperature increase. FIG. 6 (A and B) shows the inverted first derivative of fluorescence emission, where minima indicate the value of the melting temperature. Tₘ of WT-Δexo-Bst496 DNAP and its variants ranged from 75° C. (Bst496 ATMI and Bst496 TQI) to 77° C. (Bst496 D775Q=76.25° C.; Bst496 P597R=76.67° C.; and WT-Δexo-Bst496=76.83° C.) except for Bst496 A639T (Tₘ=74.17° C.) and Bst496 M7921 (Tₘ=78° C.) (Table 4; FIG. 6B). In comparison, Tₘ of Bst 2.0 WS and Bst 3.0 (both from New England Biolabs, MA) was 78.67° C. and 78° C., respectively (Table 4; FIG. 6A).

### Reverse Transcriptase Activity of Bst496 DNAP at Different Temperatures

The reverse transcriptase (RT) activity of WT-Δexo-Bst496 DNAP and its variants: Bst496 P597R (SEQ ID NO: 5), Bst496 A639T (SEQ ID NO: 6), Bst496 D775Q (SEQ ID NO: 7), Bst496 M792I (SEQ ID NO: 8), Bst496 ATMI (SEQ ID NO: 9), and Bst496 TQI (SEQ ID NO: 10) at different temperatures were evaluated in LAMP with 500 copies of SARS-COV-2 RNA. The RT-LAMP reaction was performed in 25 μL with 1× Isothermal Buffer (Varizymes; 20 mM Tris-HCl [pH 8.3], 10 mM K₂SO₄, 50 mM KCl, 2 mM MgSO₄, 0.1% Tween-20), 4 mM additional MgSO₄, 1 mM dNTP mix, 1× primer, 0.5 μg porcine RNase inhibitor, and intercalating dye (SYTO-82) for real-time monitoring of nucleic acid amplification. No secondary enzymes (RT polymerase) were added. The reactions were performed in a real-time thermocycler (CFX-96, BioRad) at 60-72° C.

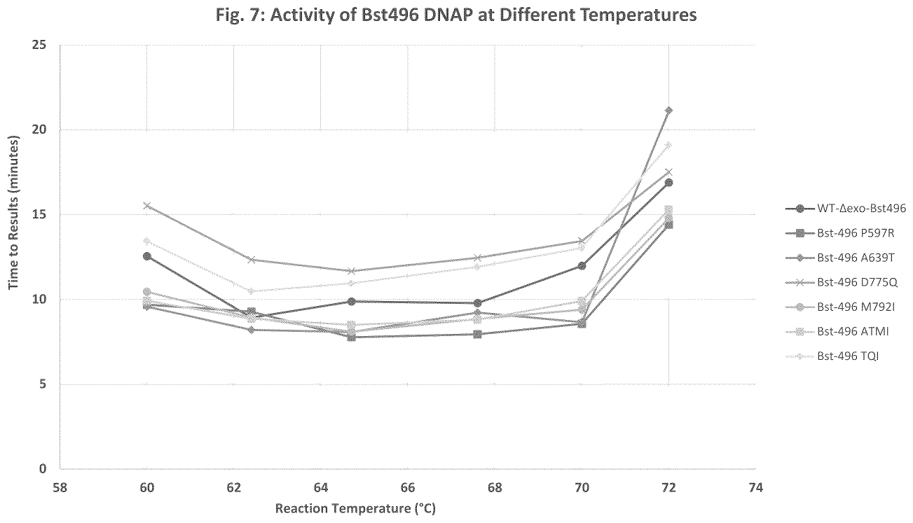

As shown in FIG. 7, all Bst496 variants had the fastest amplification at 62-68° C. Bst496 P597R, Bst496 M7921, and Bst496 ATMI were equal to or slightly faster than WT-Δexo-Bst496 at 60-72° C. Bst496 A639T was faster than WT-Δexo-Bst496 at 60-70° C., but experienced a significant drop-off at 72° C.

TABLE 2
Description of LAMP Primers

| Target | Primer | Sequence (5′ to 3′) | SEQ ID NO: |
|---|---|---|---|
| MS2 | F3 | TGTCATGGGATCCGGATGTT | 11 |
| | B3 | CAATAGAGCCGCTCTCAGAG | 12 |
| | FL | CCAGAGAGGAGGTTGCCAA | 13 |
| | BL | TGCAGGATGCAGCGCCTTA | 14 |
| | FIP | GCCCAAACAACGACGATCGGTAAAACCAGCATCCGTAGCCT | 15 |
| | BIP | GCACGTTCTCCAACGGTGCTGGTTGCTTGTTCAGCGAACT | 16 |
| SARS-COV-2 | F3 | GCGCGATCAAAACAACGTC | 17 |
| | B3 | GGCCCAGTTCCTAGGTAG | 18 |
| | FL | GTGAGAGCGGTGAACCAAG | 19 |
| | BL | TCCAATTAACACCAATAGCAGTC | 20 |
| | FIP | AAGGTCTTCCTTGCCATGTTGACCCAAGGTTTACCCAATAATAC | 21 |
| | | TGCG | |
| | BIP | AAATTCCCTCGAGGACAAGGCGTAGCTCTTCGGTAGTAGCC | 22 |
| Foot and Mouth | F3 | TGGGTTTTACAAACCTGTGATGGCT | 23 |
| disease virus | B3 | GCACACGGCGTTCACCCAAC | 24 |
| (FMDV) | FL | CCACGGCGTGCAAAGGA | 26 |
| | BL | CTGACGAGTACCGGCGTCT | 27 |
| | FIP | GCCACGGAGATCAACTTCTCCTGTACGAAGACCCTCGAGGCTA | 28 |
| | | TCCTC | |
| | BIP | AGGACTCGCCGTCCACTCTGCTTGGAATCTCAAAGAGGCCCTG | 29 |
| | | G | |
| Candida auris | F3 | TGGTTCTCGCATCGATGAAGA | 30 |
| | B3 | CGTAGATTTTTTTCGTGCAAGCT | 31 |
| | FL | TCACGTCTGCAAGTCATACT | 32 |
| | BL | TGCCTGTTTGAGCGTGAT | 33 |
| | FIP | TGTGCGTTCAAAGATTCGATGATTTTTGCAGCGAAATGCGATA | 34 |
| | | CGT | |
| | BIP | TTGCGCCTTGGGGTATTCCCCAAGTTTTTTTGTGAATGCAACGC | 35 |
| | | CACCGCG | |

TABLE 3
List of primers used for cloning and sequencing of the Bst496 DNA polymerase
and its variants

| | | SEQ |
|---|---|---|
| Description | Sequence | ID NO |
| KT496P597RBsiWIF1 | GAGGGCCTGCTGAAAGTCGTAC | 36 |
| (forward primer for introduction of P597R | GTCGCGACACCAAAAAAGTCCA | |
| mutation) | TACTATCTTTAACC | |
| KT496P597RBsiWIR1 | GGTTAAAGATAGTATGGACTTT | 37 |
| (reverse primer for introduction of P597R | TTTGGTGTCGCGACGTACGACT | |
| mutation) | TTCAGCAGGCCCTC | |
| KT496A639TAclIF1 | GAGGGCCGTAAAATCCGCCAA | 38 |
| (forward primer for introduction of A639T | ACGTTTGTTCCGAGCGAGCCGG | |
| mutation) | G | |
| KT496A639TAclIR1 | CCCGGCTCGCTCGGAACAAACG | 39 |
| (reverse primer for introduction of A639T | TTTGGCGGATTTTACGGCCCTC | |
| mutation) | | |
| KT496D775QSpeIF1 | CACCGTCGTCGTTATCTGCCGC | 40 |
| (forward primer for introduction of D775Q | AGATCACTAGTCGCAATTTCAA | |
| mutation) | CGTCCGTAG | |
| KT496D775QSpeIR1 | CTACGGACGTTGAAATTGCGAC | 41 |
| (reverse primer for introduction of D775Q | TAGTGATCTGCGGCAGATAACG | |
| mutation) | ACGACGGTG | |
| KT496M792IAseIF1 | CTTCGCGGAGCGTACGGCAATT | 42 |
| (forward primer for introduction of M792I | AATACGCCGATCCAGGGCAGCG | |
| mutation) | C | |
| KT496M792IAseIR1 | GCGCTGCCCTGGATCGGCGTAT | 43 |
| (reverse primer for introduction of M792I | TAATTGCCGTACGCTCCGCGAA | |
| mutation) | G | |
| MS2F3b | TGTCATGGGATCCGGATGTTTT | 44 |
| | ACAAACCA | |
| MS2R | ACGGCGCACATTGGTCTCGGA | 45 |
| M13FT-41 | CGCCAGGGTTTTCCCAGTCACG | 46 |
| | AC | |

TABLE 4

| Source | Bst polymerase | Tm (° C.) |
|---|---|---|
| NEB | Bst 2.0 WS | 78.67 |
| | Bst 3.0 | 78.00 |
| Varizymes | WT-Δexo-Bst496 | 76.83 |
| | Bst496 P597R | 76.67 |
| | Bst496 A639T | 74.17 |
| | Bst496 M792I | 78.00 |
| | Bst496 D775Q | 76.25 |
| | Bst496 ATMI | 75.00 |
| | Bst496 TQI | 75.00 |

TABLE 5
Nucleic acid sequences of FL-WT-Bst496 DNA polymerase (DNAP) and its
variants

| SEQ | | |
|---|---|---|
| ID NO | Description | Sequence |
| 47 | FL-WT- | TTGAAGAAAAAACTTGTCTTGATTGACGGCAACAGTGTGGCC |
| | Bst496, | TATCGCGCTTTTTTTGCGCTGCCGCTTTTGCATAACGACAAAG |
| | TWG31496 | GCGTTCATACGAATGCGGTCTACGGATTTGCCATGATGCTGCG |
| | | TAAAATCGTGGCGGAAGAGGCTCCCACTCATTTGTTAGTGGC |
| | | GTTTGATGCGGGCAAAACGACATTTCGTCATGAAGTGTTTCGC |
| | | GAGTATAAAGGCGGGCGCCAGCAGACGCCCCCGGAATTGTCC |
| | | GAGCAGTTTCCACTGCTGCGCGAGCTGTTGAACGCCTATCGC |
| | | ATTCCAGCTTACGAACTTGACAACTACGAAGCGGACGACATT |
| | | ATCGGAACGCTCGCCGCCCGCGCTGAGCAGGCCGGGTTTGAG |
| | | GTGAAAGTCATCTCCGGAGACCGGGATTTGACCCAACTCGCC |
| | | TCCCCCCATGTGACGGTGGAAATCACGAAAAAAGGGATCACC |
| | | GAGATGGAATCGTACACGCCGGAAACGATTCGCGAAAAATAC |
| | | GGGCTCGCTCCCGAGCAAATCGTTGACTTAAAAGGACTGATG |
| | | GGGGATAAATCGGACAACATCCCTGGCGTGCCCGGCATCGGC |
| | | GAAAAAACGGCGGTCAAGTTGTTAAAGCAGTTCGGGACAGTC |
| | | GAAAATGTGCTCGACTCCATTGATGAAATCCAAGGGGAAAAG |
| | | CTGAAAGAAACGCTGCGCCAACATCGCGATACCGCCCTCCTA |
| | | AGCAAACGGCTGGCGGGCATTTGCCGCGACGCTCCGATCGCC |
| | | CTGTCGCTTGAGGATACGGCCTACGAAGGCGAAGACCGGGAG |
| | | AGCGTGATCGCTTTGTTTAAAGAGCTCGGGTTTCAGTCGCTCC |
| | | TTGAGAAGATGGAAGGGCCGAGGGCGGAAGAGAGCGAGCCG |
| | | CTTGCCCACATCGACTTCACCATGGCGGAGTGCGTGACGGAA |
| | | GAGATGCTCGCCGACAAGGCGGCCTTGGTCGTTGAAGTCGTG |
| | | GAAGACAACTATCATGACGCGCCGATCGTCGGCATTGCTGTT |
| | | GTCTCGGAACGCGGGCGGTTTTTCCTGCCTTCGGAGGTGGCGC |
| | | TGGCTGACCCGCAGTTTCGCGCTTGGCTTGCCGATGAGACGA |
| | | AGAAAAAAAGCATGTTTGACTCGAAGCGGGCGACGGTCGCTT |
| | | TACGATGGAAAGGGATCGACCTGCGCGGCGTCTCGTTTGATT |
| | | TGCTGCTGGCGGCGTATTTGCTCAACCCTGGACAAGATGCCA |
| | | GCGATGTCGCGGCGGTGGCCAAAATGAAACAATACGAGGCC |
| | | GTGCGGTCCGATGAAGCGGTCTACGGCAAAGGGGCGAAACG |
| | | GTCTGTTCCGGACGAGCCGGCACTCGCCGAGCATCTCGTCCG |
| | | CAAGGCGGCGGCGATTTGGGCGCTCGAGCGGCCAATGATGGA |
| | | TGACTTGCGCCGCAACGAGCAGGACGAACTGTTGATGGCGCT |
| | | TGAGCAGCCGTTAGCGGCGATTTTGGCCGATATGGAATTCAC |
| | | CGGGGTGAAAGTGGACACCGAGCGCCTGAACCAGATGGGAC |
| | | AGGAACTCGCCGAGCAGCTGCGCGCGGCCGAACGGCGCATTT |
| | | ACGAACTGGCCGGCCAAGAATTCAATATCAATTCGCCGAAAC |
| | | AGCTCGGCGTCATTTTATTTGAAAAGCTGCAACTGCCTGTGTT |
| | | GAAAAAAACGAAGACCGGCTATTCAACATCGGCGGACGTGCT |
| | | GGAAAAGCTTGCGCCCCATCACGAGATTGTGGAGAATATTTT |
| | | GCACTACCGCCAACTAGGCAAGCTGCAGTCGACATATGTCGA |
| | | AGGATTGTTGAAGGTCGTGCGCCCCGATACGAAGAAAGTGCA |
| | | TACGATCTTCAATCAAGCGTTGACGCAAACCGGCCGCCTCAG |
| | | CTCGACGGAGCCGAACTTGCAAAACATTCCGATTCGGCTGGA |
| | | AGAAGGGCGGAAAATCCGGCAAGCGTTCGTGCCGTCCGAGCC |
| | | TGGCTGGCTCATGTTTGCGGCTGACTATTCGCAAATCGAACTG |
| | | CGCGTGTTGGCCCATATCGCCGAAGATGACAATTTAATTGAA |
| | | GCGTTTCGCCGCGATTTGGACATTCATACGAAAACGGCTATG |
| | | GACATTTTTCACGTCAAGGAAGAGGACGTCACCCCCAACATG |
| | | CGCCGGCAAGCGAAGGCCGTCAACTTTGGCATCGTCTATGGG |
| | | ATCAGCGATTACGGATTGGCGCAAAACTTAAACATTACGCGC |
| | | AAAGAGGCCGCCGAGTTTATCGAACGCTATTTTGCCAGCTTCC |
| | | CGGGCGTCAAGCGGTATATGGAAACGATCGTGCAAGAGGCG |
| | | AAACAGAAAGGATATGTCACCACGCTTTTGCATCGGCGCCGC |
| | | TATTTGCCGGACATCACGAGCCGCAACTTCAACGTCCGCAGC |
| | | TTTGCCGAGCGGACGGCGATGAATACGCCGATCCAAGGCAGC |
| | | GCCGCTGACATCATTAAAAAGGCGATGATCGATTTGAGCGCG |
| | | CGCCTGAAGGAAGAGCGGCTGCAGGCGCGCCTGTTGCTGCAA |
| | | GTGCACGACGAGCTCATTTTGGAGGCGCCTAGTGACGAAATA |
| | | GAGCGGCTGTGCCGAATCGTGCCGGAAGTGATGGAGCAAGCC |
| | | GTCACGCTCCGCGTGCCGCTGAAAGTCGATTATCATTACGGA |
| | | CCGACATGGTATGATGCGAAATAA |
| 48 | Exo, | TTGAAGAAAAAACTTGTCTTGATTGACGGCAACAGTGTGGCC |
| | exonuclease | TATCGCGCTTTTTTTGCGCTGCCGCTTTTGCATAACGACAAAG |
| | domain | GCGTTCATACGAATGCGGTCTACGGATTTGCCATGATGCTGCG |
| | | TAAAATCGTGGCGGAAGAGGCTCCCACTCATTTGTTAGTGGC |
| | | GTTTGATGCGGGCAAAACGACATTTCGTCATGAAGTGTTTCGC |
| | | GAGTATAAAGGCGGGCGCCAGCAGACGCCCCCGGAATTGTCC |
| | | GAGCAGTTTCCACTGCTGCGCGAGCTGTTGAACGCCTATCGC |
| | | ATTCCAGCTTACGAACTTGACAACTACGAAGCGGACGACATT |
| | | ATCGGAACGCTCGCCGCCCGCGCTGAGCAGGCCGGGTTTGAG |
| | | GTGAAAGTCATCTCCGGAGACCGGGATTTGACCCAACTCGCC |
| | | TCCCCCCATGTGACGGTGGAAATCACGAAAAAAGGGATCACC |
| | | GAGATGGAATCGTACACGCCGGAAACGATTCGCGAAAAATAC |
| | | GGGCTCGCTCCCGAGCAAATCGTTGACTTAAAAGGACTGATG |
| | | GGGGATAAATCGGACAACATCCCTGGCGTGCCCGGCATCGGC |
| | | GAAAAAACGGCGGTCAAGTTGTTAAAGCAGTTCGGGACAGTC |
| | | GAAAATGTGCTCGACTCCATTGATGAAATCCAAGGGGAAAAG |
| | | CTGAAAGAAACGCTGCGCCAACATCGCGATACCGCCCTCCTA |
| | | AGCAAACGGCTGGCGGGCATTTGCCGCGACGCTCCGATCGCC |
| | | CTGTCGCTTGAGGATACGGCCTACGAAGGCGAAGACCGGGAG |
| | | AGCGTGATCGCTTTGTTTAAAGAGCTCGGGTTTCAGTCGCTCC |
| | | TTGAGAAGATGGAAGGGCCGAGGGCGGAAGAGAGCGAGCCG |
| 49 | 10XHis, tag | ATGCACCACCATCATCATCACCATCACCACCAC |
| | for nickel | |
| | affinity | |
| | purification | |
| 50 | WT-Δexo- | ATGCACCACCATCATCATCACCATCACCACCACCTGGCCCAC |
| | Bst496, | ATCGACTTCACCATGGCCGAGTGCGTTACCGAAGAAATGCTG |
| | 10XHis Aexo | GCGGATAAGGCAGCGCTGGTCGTTGAAGTTGTTGAGGATAAC |
| | Bst496 | TACCACGACGCTCCGATTGTGGGTATTGCGGTCGTGAGCGAG |
| | | CGCGGTCGCTTCTTCCTGCCGAGCGAAGTCGCGCTGGCGGAT |
| | | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCTG |
| | | CGATTTGGGCGTTGGAGCGTCCTATGATGGATGACTTGCGCC |
| | | GTAACGAACAAGACGAACTGCTGATGGCGCTGGAGCAGCCGC |
| | | TGGCGGCAATCCTGGCCGACATGGAATTCACCGGTGTCAAAG |
| | | TTGACACCGAGCGTCTGAACCAGATGGGTCAAGAATTAGCGG |
| | | AGCAACTGCGTGCAGCGGAGCGCAGAATCTACGAATTGGCAG |
| | | GCCAAGAATTCAACATCAACAGCCCGAAACAGCTGGGTGTGA |
| | | TTCTGTTTGAAAAGTTACAGCTGCCGGTCCTGAAGAAAACTA |
| | | AGACCGGCTACAGCACCTCTGCAGACGTGTTGGAGAAACTGG |
| | | CCCCACACCACGAAATCGTGGAGAATATCCTGCATTACCGTC |
| | | AGCTGGGCAAGCTGCAGAGCACCTATGTTGAGGGCCTGCTGA |
| | | AAGTCGTGCGTCCGGACACCAAAAAAGTCCATACTATCTTTA |
| | | ACCAAGCGCTGACCCAGACGGGTCGTCTTAGCAGCACCGAGC |
| | | CGAATCTGCAAAACATTCCGATCCGCTTGGAAGAGGGCCGTA |
| | | AAATCCGCCAGGCCTTTGTTCCGAGCGAGCCGGGTTGGCTGA |
| | | TGTTTGCGGCTGATTATAGCCAGATCGAGTTGCGTGTGCTGGC |
| | | GCATATTGCAGAGGACGACAATCTGATTGAAGCCTTTCGTCG |
| | | CGATCTGGATATTCATACGAAAACCGCCATGGACATCTTCCA |
| | | CGTCAAAGAGGAAGATGTGACCCCGAACATGCGTCGTCAGGC |
| | | GAAAGCTGTTAATTTCGGTATTGTTTACGGCATTTCTGACTAT |
| | | GGTCTGGCCCAGAATCTGAATATTACGCGTAAAGAGGCCGCA |
| | | GAGTTTATTGAGCGTTATTTCGCGTCTTTCCCGGGTGTCAAGC |
| | | GTTACATGGAAACCATTGTGCAAGAAGCGAAGCAAAAGGGCT |
| | | ATGTGACGACCCTGTTGCACCGTCGTCGTTATCTGCCGGACAT |
| | | CACGAGCCGCAATTTCAACGTCCGTAGCTTCGCGGAGCGTAC |
| | | GGCAATGAACACGCCGATCCAGGGCAGCGCAGCGGACATCAT |
| | | CAAAAAAGCCATGATCGACCTGAGCGCACGCCTGAAAGAGG |
| | | AACGCCTGCAGGCCCGTCTGCTGCTGCAAGTGCACGATGAAC |
| | | TGATTCTGGAAGCGCCGTCCGATGAAATTGAGCGTCTGTGTC |
| | | GCATTGTTCCTGAAGTTATGGAACAAGCTGTGACCCTCCGTGT |
| | | TCCGCTGAAAGTTGACTACCACTACGGTCCGACCTGGTATGA |
| | | CGCAAAGTGA |
| 51 | Bst496 | ATGCACCACCATCATCATCACCATCACCACCACCTGGCCCAC |
| | P597R | ATCGACTTCACCATGGCCGAGTGCGTTACCGAAGAAATGCTG |
| | | GCGGATAAGGCAGCGCTGGTCGTTGAAGTTGTTGAGGATAAC |
| | | TACCACGACGCTCCGATTGTGGGTATTGCGGTCGTGAGCGAG |
| | | CGCGGTCGCTTCTTCCTGCCGAGCGAAGTCGCGCTGGCGGAT |
| | | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCGC |
| | | GATTTGGGCGTTGGAGCGTCCTATGATGGATGACTTGCGCCGT |
| | | AACGAACAAGACGAACTGCTGATGGCGCTGGAGCAGCCGCTG |
| | | GCGGCAATCCTGGCCGACATGGAATTCACCGGTGTCAAAGTT |
| | | GACACCGAGCGTCTGAACCAGATGGGTCAAGAATTAGCGGAG |
| | | CAACTGCGTGCAGCGGAGCGCAGAATCTACGAATTGGCAGGC |
| | | CAAGAATTCAACATCAACAGCCCGAAACAGCTGGGTGTGATT |
| | | CTGTTTGAAAAGTTACAGCTGCCGGTCCTGAAGAAAACTAAG |
| | | ACCGGCTACAGCACCTCTGCAGACGTGTTGGAGAAACTGGCC |
| | | CCACACCACGAAATCGTGGAGAATATCCTGCATTACCGTCAG |
| | | CTGGGCAAGCTGCAGAGCACCTATGTTGAGGGCCTGCTGAAA |
| | | GTCGTACGTCGCGACACCAAAAAAGTCCATACTATCTTTAAC |
| | | CAAGCGCTGACCCAGACGGGTCGTCTTAGCAGCACCGAGCCG |
| | | AATCTGCAAAACATTCCGATCCGCTTGGAAGAGGGCCGTAAA |
| | | ATCCGCCAGGCCTTTGTTCCGAGCGAGCCGGGTTGGCTGATGT |
| | | TTGCGGCTGATTATAGCCAGATCGAGTTGCGTGTGCTGGCGC |
| | | ATATTGCAGAGGACGACAATCTGATTGAAGCCTTTCGTCGCG |
| | | ATCTGGATATTCATACGAAAACCGCCATGGACATCTTCCACGT |
| | | CAAAGAGGAAGATGTGACCCCGAACATGCGTCGTCAGGCGA |
| | | AAGCTGTTAATTTCGGTATTGTTTACGGCATTTCTGACTATGG |
| | | TCTGGCCCAGAATCTGAATATTACGCGTAAAGAGGCCGCAGA |
| | | GTTTATTGAGCGTTATTTCGCGTCTTTCCCGGGTGTCAAGCGT |
| | | TACATGGAAACCATTGTGCAAGAAGCGAAGCAAAAGGGCTAT |
| | | GTGACGACCCTGTTGCACCGTCGTCGTTATCTGCCGGACATCA |
| | | CGAGCCGCAATTTCAACGTCCGTAGCTTCGCGGAGCGTACGG |
| | | CAATGAACACGCCGATCCAGGGCAGCGCAGCGGACATCATCA |
| | | AAAAAGCCATGATCGACCTGAGCGCACGCCTGAAAGAGGAA |
| | | CGCCTGCAGGCCCGTCTGCTGCTGCAAGTGCACGATGAACTG |
| | | ATTCTGGAAGCGCCGTCCGATGAAATTGAGCGTCTGTGTCGC |
| | | ATTGTTCCTGAAGTTATGGAACAAGCTGTGACCCTCCGTGTTC |
| | | CGCTGAAAGTTGACTACCACTACGGTCCGACCTGGTATGACG |
| | | CAAAGTGA |
| 52 | Bst496 | ATGCACCACCATCATCATCACCATCACCACCACCTGGCCCAC |
| | A639T | ATCGACTTCACCATGGCCGAGTGCGTTACCGAAGAAATGCTG |
| | | GCGGATAAGGCAGCGCTGGTCGTTGAAGTTGTTGAGGATAAC |
| | | TACCACGACGCTCCGATTGTGGGTATTGCGGTCGTGAGCGAG |
| | | CGCGGTCGCTTCTTCCTGCCGAGCGAAGTCGCGCTGGCGGAT |
| | | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCTG |
| | | CGATTTGGGCGTTGGAGCGTCCTATGATGGATGACTTGCGCC |
| | | GTAACGAACAAGACGAACTGCTGATGGCGCTGGAGCAGCCGC |
| | | TGGCGGCAATCCTGGCCGACATGGAATTCACCGGTGTCAAAG |
| | | TTGACACCGAGCGTCTGAACCAGATGGGTCAAGAATTAGCGG |
| | | AGCAACTGCGTGCAGCGGAGCGCAGAATCTACGAATTGGCAG |
| | | GCCAAGAATTCAACATCAACAGCCCGAAACAGCTGGGTGTGA |
| | | TTCTGTTTGAAAAGTTACAGCTGCCGGTCCTGAAGAAAACTA |
| | | AGACCGGCTACAGCACCTCTGCAGACGTGTTGGAGAAACTGG |
| | | CCCCACACCACGAAATCGTGGAGAATATCCTGCATTACCGTC |
| | | AGCTGGGCAAGCTGCAGAGCACCTATGTTGAGGGCCTGCTGA |
| | | AAGTCGTGCGTCCGGACACCAAAAAAGTCCATACTATCTTTA |
| | | ACCAAGCGCTGACCCAGACGGGTCGTCTTAGCAGCACCGAGC |
| | | CGAATCTGCAAAACATTCCGATCCGCTTGGAAGAGGGCCGTA |
| | | AAATCCGCCAAACGTTTGTTCCGAGCGAGCCGGGTTGGCTGA |
| | | TGTTTGCGGCTGATTATAGCCAGATCGAGTTGCGTGTGCTGGC |
| | | GCATATTGCAGAGGACGACAATCTGATTGAAGCCTTTCGTCG |
| | | CGATCTGGATATTCATACGAAAACCGCCATGGACATCTTCCA |
| | | CGTCAAAGAGGAAGATGTGACCCCGAACATGCGTCGTCAGGC |
| | | GAAAGCTGTTAATTTCGGTATTGTTTACGGCATTTCTGACTAT |
| | | GGTCTGGCCCAGAATCTGAATATTACGCGTAAAGAGGCCGCA |
| | | GAGTTTATTGAGCGTTATTTCGCGTCTTTCCCGGGTGTCAAGC |
| | | GTTACATGGAAACCATTGTGCAAGAAGCGAAGCAAAAGGGCT |
| | | ATGTGACGACCCTGTTGCACCGTCGTCGTTATCTGCCGGACAT |
| | | CACGAGCCGCAATTTCAACGTCCGTAGCTTCGCGGAGCGTAC |
| | | GGCAATGAACACGCCGATCCAGGGCAGCGCAGCGGACATCAT |
| | | CAAAAAAGCCATGATCGACCTGAGCGCACGCCTGAAAGAGG |
| | | AACGCCTGCAGGCCCGTCTGCTGCTGCAAGTGCACGATGAAC |
| | | TGATTCTGGAAGCGCCGTCCGATGAAATTGAGCGTCTGTGTC |
| | | GCATTGTTCCTGAAGTTATGGAACAAGCTGTGACCCTCCGTGT |
| | | TCCGCTGAAAGTTGACTACCACTACGGTCCGACCTGGTATGA |
| | | CGCAAAGTGA |
| 53 | Bst496 | ATGCACCACCATCATCATCACCATCACCACCACCTGGCCCAC |
| | D775Q | ATCGACTTCACCATGGCCGAGTGCGTTACCGAAGAAATGCTG |
| | | GCGGATAAGGCAGCGCTGGTCGTTGAAGTTGTTGAGGATAAC |
| | | TACCACGACGCTCCGATTGTGGGTATTGCGGTCGTGAGCGAG |
| | | CGCGGTCGCTTCTTCCTGCCGAGCGAAGTCGCGCTGGCGGAT |
| | | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCTG |
| | | CGATTTGGGCGTTGGAGCGTCCTATGATGGATGACTTGCGCC |
| | | GTAACGAACAAGACGAACTGCTGATGGCGCTGGAGCAGCCGC |
| | | TGGCGGCAATCCTGGCCGACATGGAATTCACCGGTGTCAAAG |
| | | TTGACACCGAGCGTCTGAACCAGATGGGTCAAGAATTAGCGG |
| | | AGCAACTGCGTGCAGCGGAGCGCAGAATCTACGAATTGGCAG |
| | | GCCAAGAATTCAACATCAACAGCCCGAAACAGCTGGGTGTGA |
| | | TTCTGTTTGAAAAGTTACAGCTGCCGGTCCTGAAGAAAACTA |
| | | AGACCGGCTACAGCACCTCTGCAGACGTGTTGGAGAAACTGG |
| | | CCCCACACCACGAAATCGTGGAGAATATCCTGCATTACCGTC |
| | | AGCTGGGCAAGCTGCAGAGCACCTATGTTGAGGGCCTGCTGA |
| | | AAGTCGTGCGTCCGGACACCAAAAAAGTCCATACTATCTTTA |
| | | ACCAAGCGCTGACCCAGACGGGTCGTCTTAGCAGCACCGAGC |
| | | CGAATCTGCAAAACATTCCGATCCGCTTGGAAGAGGGCCGTA |
| | | AAATCCGCCAGGCCTTTGTTCCGAGCGAGCCGGGTTGGCTGA |
| | | TGTTTGCGGCTGATTATAGCCAGATCGAGTTGCGTGTGCTGGC |
| | | GCATATTGCAGAGGACGACAATCTGATTGAAGCCTTTCGTCG |
| | | CGATCTGGATATTCATACGAAAACCGCCATGGACATCTTCCA |
| | | CGTCAAAGAGGAAGATGTGACCCCGAACATGCGTCGTCAGGC |
| | | GAAAGCTGTTAATTTCGGTATTGTTTACGGCATTTCTGACTAT |
| | | GGTCTGGCCCAGAATCTGAATATTACGCGTAAAGAGGCCGCA |
| | | GAGTTTATTGAGCGTTATTTCGCGTCTTTCCCGGGTGTCAAGC |
| | | GTTACATGGAAACCATTGTGCAAGAAGCGAAGCAAAAGGGCT |
| | | ATGTGACGACCCTGTTGCACCGTCGTCGTTATCTGCCGCAGAT |
| | | CACTAGTCGCAATTTCAACGTCCGTAGCTTCGCGGAGCGTAC |
| | | GGCAATGAACACGCCGATCCAGGGCAGCGCAGCGGACATCAT |
| | | CAAAAAAGCCATGATCGACCTGAGCGCACGCCTGAAAGAGG |
| | | AACGCCTGCAGGCCCGTCTGCTGCTGCAAGTGCACGATGAAC |
| | | TGATTCTGGAAGCGCCGTCCGATGAAATTGAGCGTCTGTGTC |
| | | GCATTGTTCCTGAAGTTATGGAACAAGCTGTGACCCTCCGTGT |
| | | TCCGCTGAAAGTTGACTACCACTACGGTCCGACCTGGTATGA |
| | | CGCAAAGTGA |
| 54 | Bst496 | ATGCACCACCATCATCATCACCATCACCACCACCTGGCCCAC |
| | M792I | ATCGACTTCACCATGGCCGAGTGCGTTACCGAAGAAATGCTG |
| | | GCGGATAAGGCAGCGCTGGTCGTTGAAGTTGTTGAGGATAAC |
| | | TACCACGACGCTCCGATTGTGGGTATTGCGGTCGTGAGCGAG |
| | | CGCGGTCGCTTCTTCCTGCCGAGCGAAGTCGCGCTGGCGGAT |
| | | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCTG |
| | | CGATTTGGGCGTTGGAGCGTCCTATGATGGATGACTTGCGCC |
| | | GTAACGAACAAGACGAACTGCTGATGGCGCTGGAGCAGCCGC |
| | | TGGCGGCAATCCTGGCCGACATGGAATTCACCGGTGTCAAAG |
| | | TTGACACCGAGCGTCTGAACCAGATGGGTCAAGAATTAGCGG |
| | | AGCAACTGCGTGCAGCGGAGCGCAGAATCTACGAATTGGCAG |
| | | GCCAAGAATTCAACATCAACAGCCCGAAACAGCTGGGTGTGA |
| | | TTCTGTTTGAAAAGTTACAGCTGCCGGTCCTGAAGAAAACTA |
| | | AGACCGGCTACAGCACCTCTGCAGACGTGTTGGAGAAACTGG |
| | | CCCCACACCACGAAATCGTGGAGAATATCCTGCATTACCGTC |
| | | AGCTGGGCAAGCTGCAGAGCACCTATGTTGAGGGCCTGCTGA |
| | | AAGTCGTGCGTCCGGACACCAAAAAAGTCCATACTATCTTTA |
| | | ACCAAGCGCTGACCCAGACGGGTCGTCTTAGCAGCACCGAGC |
| | | CGAATCTGCAAAACATTCCGATCCGCTTGGAAGAGGGCCGTA |
| | | AAATCCGCCAGGCCTTTGTTCCGAGCGAGCCGGGTTGGCTGA |
| | | TGTTTGCGGCTGATTATAGCCAGATCGAGTTGCGTGTGCTGGC |
| | | GCATATTGCAGAGGACGACAATCTGATTGAAGCCTTTCGTCG |
| | | CGATCTGGATATTCATACGAAAACCGCCATGGACATCTTCCA |
| | | CGTCAAAGAGGAAGATGTGACCCCGAACATGCGTCGTCAGGC |
| | | GAAAGCTGTTAATTTCGGTATTGTTTACGGCATTTCTGACTAT |
| | | GGTCTGGCCCAGAATCTGAATATTACGCGTAAAGAGGCCGCA |
| | | GAGTTTATTGAGCGTTATTTCGCGTCTTTCCCGGGTGTCAAGC |
| | | GTTACATGGAAACCATTGTGCAAGAAGCGAAGCAAAAGGGCT |
| | | ATGTGACGACCCTGTTGCACCGTCGTCGTTATCTGCCGGACAT |
| | | CACGAGCCGCAATTTCAACGTCCGTAGCTTCGCGGAGCGTAC |
| | | GGCAATTAATACGCCGATCCAGGGCAGCGCAGCGGACATCAT |
| | | CAAAAAAGCCATGATCGACCTGAGCGCACGCCTGAAAGAGG |
| | | AACGCCTGCAGGCCCGTCTGCTGCTGCAAGTGCACGATGAAC |
| | | TGATTCTGGAAGCGCCGTCCGATGAAATTGAGCGTCTGTGTC |
| | | GCATTGTTCCTGAAGTTATGGAACAAGCTGTGACCCTCCGTGT |
| | | TCCGCTGAAAGTTGACTACCACTACGGTCCGACCTGGTATGA |
| | | CGCAAAGTGA |
| 55 | Bst496 | ATGCACCACCATCATCATCACCATCACCACCACCTGGCCCAC |
| | A639T; | ATCGACTTCACCATGGCCGAGTGCGTTACCGAAGAAATGCTG |
| | M792I, | GCGGATAAGGCAGCGCTGGTCGTTGAAGTTGTTGAGGATAAC |
| | “Bst496 | TACCACGACGCTCCGATTGTGGGTATTGCGGTCGTGAGCGAG |
| | ATMI” | CGCGGTCGCTTCTTCCTGCCGAGCGAAGTCGCGCTGGCGGAT |
| | | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCTG |
| | | CGATTTGGGCGTTGGAGCGTCCTATGATGGATGACTTGCGCC |
| | | GTAACGAACAAGACGAACTGCTGATGGCGCTGGAGCAGCCGC |
| | | TGGCGGCAATCCTGGCCGACATGGAATTCACCGGTGTCAAAG |
| | | TTGACACCGAGCGTCTGAACCAGATGGGTCAAGAATTAGCGG |
| | | AGCAACTGCGTGCAGCGGAGCGCAGAATCTACGAATTGGCAG |
| | | GCCAAGAATTCAACATCAACAGCCCGAAACAGCTGGGTGTGA |
| | | TTCTGTTTGAAAAGTTACAGCTGCCGGTCCTGAAGAAAACTA |
| | | AGACCGGCTACAGCACCTCTGCAGACGTGTTGGAGAAACTGG |
| | | CCCCACACCACGAAATCGTGGAGAATATCCTGCATTACCGTC |
| | | AGCTGGGCAAGCTGCAGAGCACCTATGTTGAGGGCCTGCTGA |
| | | AAGTCGTGCGTCCGGACACCAAAAAAGTCCATACTATCTTTA |
| | | ACCAAGCGCTGACCCAGACGGGTCGTCTTAGCAGCACCGAGC |
| | | CGAATCTGCAAAACATTCCGATCCGCTTGGAAGAGGGCCGTA |
| | | AAATCCGCCAAACGTTTGTTCCGAGCGAGCCGGGTTGGCTGA |
| | | TGTTTGCGGCTGATTATAGCCAGATCGAGTTGCGTGTGCTGGC |
| | | GCATATTGCAGAGGACGACAATCTGATTGAAGCCTTTCGTCG |
| | | CGATCTGGATATTCATACGAAAACCGCCATGGACATCTTCCA |
| | | CGTCAAAGAGGAAGATGTGACCCCGAACATGCGTCGTCAGGC |
| | | GAAAGCTGTTAATTTCGGTATTGTTTACGGCATTTCTGACTAT |
| | | GGTCTGGCCCAGAATCTGAATATTACGCGTAAAGAGGCCGCA |
| | | GAGTTTATTGAGCGTTATTTCGCGTCTTTCCCGGGTGTCAAGC |
| | | GTTACATGGAAACCATTGTGCAAGAAGCGAAGCAAAAGGGCT |
| | | ATGTGACGACCCTGTTGCACCGTCGTCGTTATCTGCCGGACAT |
| | | CACGAGCCGCAATTTCAACGTCCGTAGCTTCGCGGAGCGTAC |
| | | GGCAATTAATACGCCGATCCAGGGCAGCGCAGCGGACATCAT |
| | | CAAAAAAGCCATGATCGACCTGAGCGCACGCCTGAAAGAGG |
| | | AACGCCTGCAGGCCCGTCTGCTGCTGCAAGTGCACGATGAAC |
| | | TGATTCTGGAAGCGCCGTCCGATGAAATTGAGCGTCTGTGTC |
| | | GCATTGTTCCTGAAGTTATGGAACAAGCTGTGACCCTCCGTGT |
| | | TCCGCTGAAAGTTGACTACCACTACGGTCCGACCTGGTATGA |
| | | CGCAAAGTGA |
| 56 | Bst496 | ATGCACCACCATCATCATCACCATCACCACCACCTGGCCCAC |
| | A639T; | ATCGACTTCACCATGGCCGAGTGCGTTACCGAAGAAATGCTG |
| | D775Q; | GCGGATAAGGCAGCGCTGGTCGTTGAAGTTGTTGAGGATAAC |
| | M792I, | TACCACGACGCTCCGATTGTGGGTATTGCGGTCGTGAGCGAG |
| | “Bst496 | CGCGGTCGCTTCTTCCTGCCGAGCGAAGTCGCGCTGGCGGAT |
| | TQI” | CCACAGTTTCGCGCATGGCTGGCGGATGAGACTAAAAAGAAA |
| | | TCCATGTTTGATAGCAAGCGCGCGACGGTTGCGCTGCGTTGG |
| | | AAGGGTATCGATCTGCGTGGTGTGAGCTTCGATCTGCTCTTGG |
| | | CGGCTTACTTGCTGAATCCGGGCCAAGACGCGTCCGACGTTG |
| | | CAGCGGTGGCGAAGATGAAACAGTACGAAGCTGTCCGTTCGG |
| | | ATGAAGCCGTATATGGTAAGGGTGCAAAGCGCAGCGTCCCGG |
| | | ATGAGCCGGCATTGGCAGAGCATCTGGTGCGTAAAGCTGCTG |
| | | CGATTTGGGCGTTGGAGCGTCCTATGATGGATGACTTGCGCC |
| | | GTAACGAACAAGACGAACTGCTGATGGCGCTGGAGCAGCCGC |
| | | TGGCGGCAATCCTGGCCGACATGGAATTCACCGGTGTCAAAG |
| | | TTGACACCGAGCGTCTGAACCAGATGGGTCAAGAATTAGCGG |
| | | AGCAACTGCGTGCAGCGGAGCGCAGAATCTACGAATTGGCAG |
| | | GCCAAGAATTCAACATCAACAGCCCGAAACAGCTGGGTGTGA |
| | | TTCTGTTTGAAAAGTTACAGCTGCCGGTCCTGAAGAAAACTA |
| | | AGACCGGCTACAGCACCTCTGCAGACGTGTTGGAGAAACTGG |
| | | CCCCACACCACGAAATCGTGGAGAATATCCTGCATTACCGTC |
| | | AGCTGGGCAAGCTGCAGAGCACCTATGTTGAGGGCCTGCTGA |
| | | AAGTCGTGCGTCCGGACACCAAAAAAGTCCATACTATCTTTA |
| | | ACCAAGCGCTGACCCAGACGGGTCGTCTTAGCAGCACCGAGC |
| | | CGAATCTGCAAAACATTCCGATCCGCTTGGAAGAGGGCCGTA |
| | | AAATCCGCCAAACGTTTGTTCCGAGCGAGCCGGGTTGGCTGA |
| | | TGTTTGCGGCTGATTATAGCCAGATCGAGTTGCGTGTGCTGGC |
| | | GCATATTGCAGAGGACGACAATCTGATTGAAGCCTTTCGTCG |
| | | CGATCTGGATATTCATACGAAAACCGCCATGGACATCTTCCA |
| | | CGTCAAAGAGGAAGATGTGACCCCGAACATGCGTCGTCAGGC |
| | | GAAAGCTGTTAATTTCGGTATTGTTTACGGCATTTCTGACTAT |
| | | GGTCTGGCCCAGAATCTGAATATTACGCGTAAAGAGGCCGCA |
| | | GAGTTTATTGAGCGTTATTTCGCGTCTTTCCCGGGTGTCAAGC |
| | | GTTACATGGAAACCATTGTGCAAGAAGCGAAGCAAAAGGGCT |
| | | ATGTGACGACCCTGTTGCACCGTCGTCGTTATCTGCCGCAGAT |
| | | CACTAGTCGCAATTTCAACGTCCGTAGCTTCGCGGAGCGTAC |
| | | GGCAATTAATACGCCGATCCAGGGCAGCGCAGCGGACATCAT |
| | | CAAAAAAGCCATGATCGACCTGAGCGCACGCCTGAAAGAGG |
| | | AACGCCTGCAGGCCCGTCTGCTGCTGCAAGTGCACGATGAAC |
| | | TGATTCTGGAAGCGCCGTCCGATGAAATTGAGCGTCTGTGTC |
| | | GCATTGTTCCTGAAGTTATGGAACAAGCTGTGACCCTCCGTGT |
| | | TCCGCTGAAAGTTGACTACCACTACGGTCCGACCTGGTATGA |
| | | CGCAAAGTGA |

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

1. A Bst variant polypeptide having an amino acid sequence selected from SEQ ID Nos: 5-10 or a Bst variant polypeptide having at least one mutation of the amino acid sequence shown in SEQ ID NO: 1, wherein said mutation is selected from the group consisting of P597R, A639T, D775Q, and M792I.

2. (canceled)

3. The variant polypeptide of claim 1, wherein said polypeptide lacks an exonuclease domain.

4. The variant polypeptide of claim 1, wherein said variant exhibits increased reverse transcriptase activity relative to wild type Bst.

5. The variant polypeptide of claim 4, wherein said reverse transcriptase activity is increased 50% relative to wild type Bst.

6. The variant polypeptide of claim 4, wherein said reverse transcriptase activity is increased 100% relative to wild type Bst.

7. The variant polypeptide of claim 4, wherein said reverse transcriptase activity is increased 200% relative to wild type Bst.

8. A composition, kit, or reaction mixture comprising the variant polypeptide of claim 1.

9. The composition, kit, or reaction mixture of claim 8, wherein said composition or reaction mixture comprises one or more buffers.

10. The composition, kit, or reaction mixture of claim 9, wherein said buffer comprises Tris-HCl, (NH₄)₂SO₄, KCl, MgSO₄, and Triton® X-100 detergent.

11. The composition, kit, or reaction mixture of claim 8, further comprising a reverse transcriptase enzyme.

12. The composition, kit, or reaction mixture of claim 8, further comprising one or more additional components selected from the group consisting of nucleic acid primers, dNTPs, dyes, ions, and nucleic acid controls.

13. The composition, kit, or reaction mixture of claim 8, wherein the pH of said composition, kit or reaction mixture is between 8 and 9.

14. The composition, kit, or reaction mixture of claim 13, wherein said pH is 8.8.

15. (canceled)

16. A method of amplifying a target nucleic acid, comprising:
a) contacting a sample with the polypeptide of claim 1; and
b) performing an amplification assay on said sample.

17. (canceled)

18. The method of claim 16, wherein said amplification assay is an isothermal amplification assay.

19. The method of claim 18, wherein said isothermal amplification assay is loop-mediated isothermal amplification (LAMP).

20-21. (canceled)
